(19) United States
(12) Patent Application Publication
(10) Pub. No.: US 2023/0135456 A1
(43) Pub. Date: May 4, 2023

(54) CELL-DERIVED VESICLES COMPRISING WILD-TYPE P53 PROTEIN FOR ANTIVIRAL THERAPY

(71) Applicant: Exoprother Medical Ltd., Haifa (IL)

(72) Inventors: Alexander TENDLER, Haifa (IL); Yevgeny TENDLER, Shlomi (IL); Lana VOLOKH, Haifa (IL)

(73) Assignee: Exoprother Medical Ltd., Haifa (IL)

(21) Appl. No.: 17/918,573

(22) PCT Filed: Apr. 13, 2021
(86) PCT No.: PCT/IL2021/050424

§ 371 Date: Oct. 13, 2022

Related U.S. Application Data
(60) Provisional application No. 63/008,894, filed on Apr. 13, 2020.

(51) Int. Cl. A61K 38/17; A61K 35/30; A61P 31/14

(52) U.S. Cl. CPC A61K 38/1758; A61K 35/30; A61P 31/14

(57) ABSTRACT

A method of treating a viral infection in a subject in need thereof is disclosed. The method comprising administering to the subject a therapeutically effective amount of cell-derived vesicles comprising wild-type p53. Methods of inducing cell cycle arrest and/or apoptosis of a virally infected cell are also disclosed.

### RELATED APPLICATION/S

This application claims the benefit of priority of U.S. Provisional Patent Application No. 63/008,894 filed on Apr. 13, 2020, the contents of which are incorporated herein by reference in their entirety.

### FIELD AND BACKGROUND OF THE INVENTION

The present invention, in some embodiments thereof, relates to cell-derived vesicles comprising wild-type p53 protein and, more particularly, but not exclusively, to the use of same in treatment of viral infections.

p53 is a nuclear transcription factor which plays a major role in apoptosis, cell cycle arrest and senescence. p53 is one of the major genes responsible for maintenance of genomic stability and prevention of genome mutations in vertebrates as well as in Diptera. The p53 gene is typically classified as a tumor suppressor gene. Inactivation of p53 functions is an almost universal feature of human cancer cells. Numerous studies have shown that restoring p53 function to p53-deficient cancer cells induces growth arrest and apoptosis [Lane D. et al., Cold Spring Harb Perspect Biol (2010) 2(9): a001222].

In addition to its role as a tumor suppressor, p53 protein also plays a role in the innate immune response activated as a result of various tumor-promoting and non-tumor-promoting viral infections such as those caused by Papilloma virus, Influenza virus, Smallpox and Vaccinia viruses, Zika virus, West Nile virus, Japanese encephalitis virus, Human Immunodeficiency Virus Type 1, Human herpes simplex virus-1 and more [Aloni-Grinstein R. et al., Cancers (Basel) (2018) 10(6): 178]. Activation of p53 is affected by various cellular receptors and sensors depending on the virus type. For example, replication of viruses, especially RNA viruses, can induce type I interferons (IFNs), triggered by the production of dsRNA, which in turn induces transcription of the p53 gene [Sato and Tsurumi, Rev. Med. Virol. (2013) 23: 213-220]. Conversely, DNA viruses activate DNA damage signaling, triggered by the production of viral DNA genomes, which leads to activation of p53 [Sato and Tsurumi (2013), supra]. It should be noted that some DNA viruses, such as HSV-1/2 and adenoviruses, also induce the antiviral innate immune response which leads to type I IFN production, while some RNA viruses, such as retroviruses, activate the DNA damage response [Sato and Tsurumi (2013), supra].

p53 controls the expression of diverse target genes associated with host innate defense system. Thus, following a viral infection, p53 protein can trigger virus-induced cell cycle arrest and/or apoptosis (which inhibits the further spread of infectious pathogens) and enforce type-1 IFN antiviral response [Munoz-Fontela et al. J Exp Med (2008) 205 (8): 1929-1938] (illustrated in FIG. 1). p53 also directly transactivates the expression of several innate immunity-related genes such as IRF9, TRL3, ISG15, and MCP-1 [Sato and Tsurumi (2013), supra]. Viruses, in turn, have evolved elaborate mechanisms to subvert p53-mediated host immune responses. Accordingly, some viruses express proteins that directly suppress p53, such as Vesicular stomatitis virus (VSV) and Hepatitis C virus (HCV) [Sato and Tsurumi (2013), supra], whereas other viruses alter the regulation of p53 in an indirect manner, for example by stabilization of the p53 negative regulator MDM2, such as by coronaviruses [Lin Yuan et al., J Biol Chem (2015) 290(5): 3172-3182] (illustrated in FIG. 3), by stabilization or recruitment of proteins participating in p53 ubiquitination which lead to p53 proteasomal degradation, such as human papilloma virus or coronaviruses [Sato and Tsurumi (2013), supra; Yue Ma-Lauer et al., PNAS (2016) 113 (35): E5192-E5201] (illustrated in FIG. 2), or by sequestration of p53 from the nucleus to the cytoplasm [Sato and Tsurumi (2013), supra]. In all cases, the basic p53 function is compromised.

The development of antiviral therapies utilizing enhancement of p53 functions as a host resistance factor against virus infection has been previously discussed.

U.S. Patent Application No. 2018/0360952 relates to drug delivery systems comprising multilamellar lipid vesicles and comprising terminal-cysteine-bearing antigens or cysteine-modified antigens, such as p53, at their surface and/or internally, and the use of same for therapy.

U.S. Patent Application No. 2017/0246288 relates to drug delivery systems comprising multilamellar lipid vesicles having crosslinked lipid bilayers covalently conjugated to an agent (e.g., p53), the conjugated agent may be encapsulated within the vesicle, and the use of same for therapy.

Additional background art includes U.S. Patent Application No. 2020/071373.

### SUMMARY OF THE INVENTION

According to an aspect of some embodiments of the present invention there is provided a method of treating a viral infection in a subject in need thereof, the method comprising administering to the subject a therapeutically effective amount of cell-derived vesicles comprising wild-type p53, thereby treating the viral infection in the subject.

According to an aspect of some embodiments of the present invention there is provided a method of inducing cell cycle arrest and/or apoptosis of a virally infected cell, the method comprising contacting the cell with an effective amount of cell-derived vesicles comprising wild-type p53.

According to an aspect of some embodiments of the present invention there is provided a therapeutically effective amount of cell-derived vesicles comprising wild-type p53 for use in treating a viral infection in a subject in need thereof.

According to an aspect of some embodiments of the present invention there is provided a therapeutically effective amount of cell-derived vesicles comprising wild-type p53 for use in inducing cell cycle arrest and/or apoptosis of a virally infected cell.

According to some embodiments of the invention, the virally infected cell has been infected by a virus selected from the group consisting of a Coronavirus, an Adenovirus, a Bocavirus, a Dengue fever virus, an Ebola virus, an Enterovirus, an Epstein-Barr virus, a Human Immunodeficiency Virus (HIV), a Human herpes simplex virus (HSV), a Hantavirus, a Hepatitis B, C, D or E virus, an Influenza virus, an infectious bronchitis virus (IBV), a Japanese encephalitis virus, a Marburg virus, a Metapneumovirus, a Parvovirus, a Parainfluenza virus, a Papilloma virus, a Retrovirus, a Rabies virus, a Respiratory syncytial virus, a Rotavirus, a Rhinovirus, a Smallpox virus, a Variola virus, a Vaccinia virus, a West Nile virus, and, a Yellow fever virus, and Zika virus.

According to some embodiments of the invention, the viral infection is caused by a RNA virus.

According to some embodiments of the invention, the viral infection is caused by a DNA virus.

According to some embodiments of the invention, the viral infection is caused by a virus selected from the group consisting of a Coronavirus, an Adenovirus, a Bocavirus, a Dengue fever virus, an Ebola virus, an Enterovirus, an Epstein-Barr virus, a Human Immunodeficiency Virus (HIV), a Human herpes simplex virus (HSV), a Hantavirus, a Hepatitis B, C, D or E virus, an Influenza virus, an infectious bronchitis virus (IBV), a Japanese encephalitis virus, a Marburg virus, a Metapneumovirus, a Parvovirus, a Parainfluenza virus, a Papilloma virus, a Retrovirus, a Rabies virus, a Respiratory syncytial virus, a Rotavirus, a Rhinovirus, a Smallpox virus, a Variola virus, a Vaccinia virus, a West Nile virus, and, a Yellow fever virus, and Zika virus.

According to some embodiments of the invention, the viral infection is caused by a Coronavirus.

According to some embodiments of the invention, the Coronavirus is a severe acute respiratory syndrome coronavirus 2 (SARS-CoV-2), a Middle East respiratory syndrome coronavirus (MERS-CoV) or a severe acute respiratory syndrome coronavirus (SARS-CoV).

According to some embodiments of the invention, the cell-derived vesicles comprise cell-secreted vesicles.

According to some embodiments of the invention, the cell-derived vesicles have a mean particle diameter of about 20 to about 250 nm.

According to some embodiments of the invention, the cell-derived vesicles have a mean particle diameter of about 20 to about 200 nm.

According to some embodiments of the invention, the cell-derived vesicles comprise exosomes.

According to some embodiments of the invention, the cell-derived vesicles are essentially devoid of intact cells.

According to some embodiments of the invention, the cell expresses at least 0.001% endogenous wild-type p53 protein of the total cellular proteins and does not express recombinant p53 protein.

According to some embodiments of the invention, the cell expresses endogenous MDM2 polypeptide at a level not exceeding 0.5% of the total cellular proteins.

According to some embodiments of the invention, the cell is a cell of an animal or a human tissue.

According to some embodiments of the invention, the animal tissue is selected from the group consisting of an eye tissue, a brain tissue, a testicle tissue, a skin tissue and an intestinal tissue.

According to some embodiments of the invention, the tissue is an epidermis tissue or an epithelium of small intestines tissue.

According to some embodiments of the invention, the animal tissue comprises an eye tissue.

According to some embodiments of the invention, the eye tissue comprises a corneal epithelium tissue.

According to some embodiments of the invention, the corneal epithelium tissue comprises corneal epithelial cells.

According to some embodiments of the invention, the animal tissue comprises a testicular tissue.

According to some embodiments of the invention, the cell is selected from the group consisting of a corneal epithelium cell, an intestinal epithelial cell, a goblet cell, a cerebellum cell, a hippocampus cell, a hypothalamus cell, a pons cell, a thalamus cell, a testicular cell and an upper cerebral spine cell.

According to some embodiments of the invention, the cell is a healthy cell.

According to some embodiments of the invention, the cell is a genetically non-modified cell.

According to some embodiments of the invention, the cell is a genetically modified cells.

According to some embodiments of the invention, the cell has been treated with a MDM2 inhibitor.

According to some embodiments of the invention, the cell has been treated with a DNA damaging agent to activate the wild-type p53 protein.

According to some embodiments of the invention, the DNA damaging agent is selected from the group consisting of a UV irradiation, a gamma irradiation, a chemotherapy, an oxidative stress, hypoxia, nutrient deprivation.

According to some embodiments of the invention, the wild-type p53 comprises phosphorylated wild-type p53.

According to some embodiments of the invention, an outer surface of the cell-derived vesicles comprise a heterologous moiety for targeted delivery of the cell-derived vesicles to a target cell.

According to some embodiments of the invention, the target cell comprises a virally infected cell.

According to some embodiments of the invention, the heterologous moiety is selected from the group consisting of a protein, a peptide and a glycolipid molecule.

According to some embodiments of the invention, the method is effected ex vivo.

According to some embodiments of the invention, the method is effected in vivo.

According to some embodiments of the invention, the administering comprises a route selected from the group consisting of inhalation, intranasal, intravenous, intra-arterial, intra-tumoral, subcutaneous, intramuscular, transdermal and intraperitoneal.

According to some embodiments of the invention, the cell-derived vesicles are formulated for inhalation, intranasal, intravenous, intra-arterial, intratumoral, subcutaneous, intramuscular, transdermal or intraperitoneal mode of administration.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of p53 induction in response to viral infection as a downstream transcriptional target of type I interferon (IFN) signaling. IFN activated by viral infection acts as transcriptional activator of p53 gene. p53 contributes to virus-induced apoptosis, thereby suppressing the ability of a wide range of viruses to replicate and spread. Furthermore, p53 contributes to an increase in IFN release from infected cells, producing positive regulatory feedback loop. Previously discussed in Rivas et al. Viruses 2010, 2, 298-313.

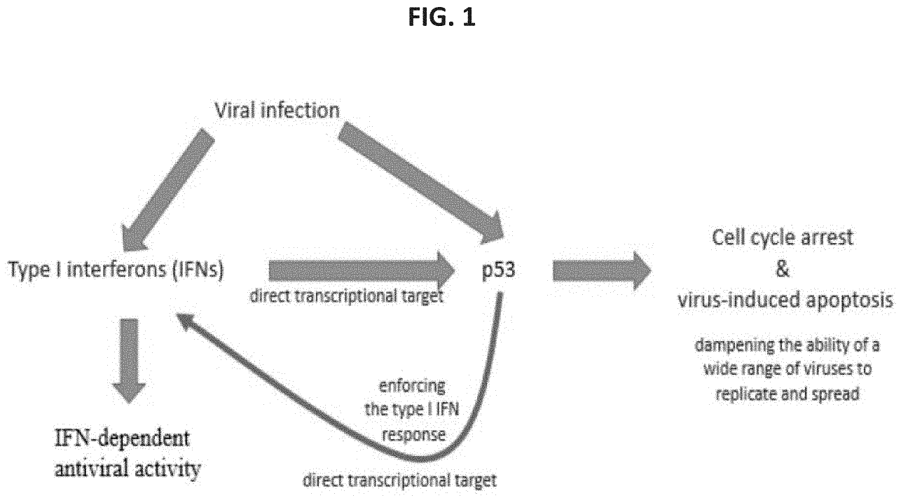

FIG. 2 is a schematic illustration of viral induced p53 suppression via ubiquitin-protein ligases (E3s). p53 is targeted by the SARS-unique domain and papain-like protease (PLpro) via the E3 ubiquitin ligase RCHY1 (PIRH2). CoVids physically interact with and stabilize E3 ubiquitin ligase ring-finger and CHY zinc-finger domain-containing 1 (RCHY1) augmenting proteasomal degradation of p53. Previously discussed in Yue Ma-Lauer et al., PNAS (2016), supra.

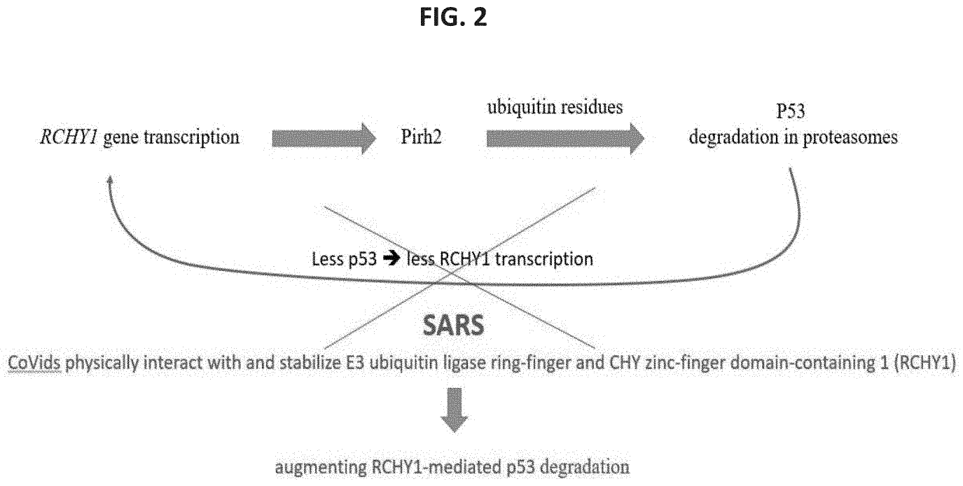

FIG. 3 is a schematic illustration of coronavirus papain-like protease (PLP2)-induced degradation of p53 through stabilizing MDM2. Previously discussed in Lin Yuan et al., J Biol Chem (2015), supra.

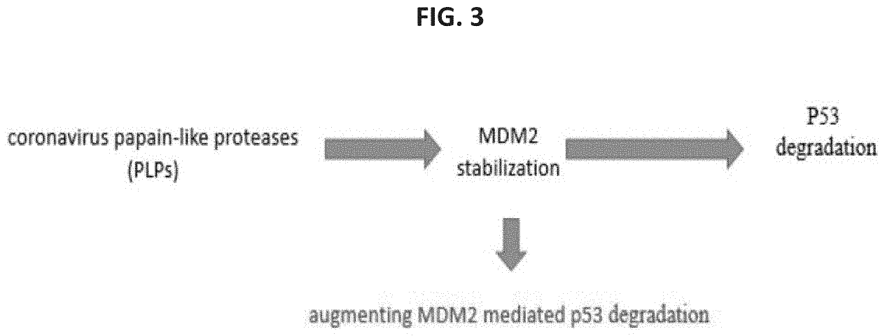

FIG. 4 is a schematic illustration of vicious cycle of p53 degradation and viral spread. Previously discussed in Lin Yuan et al., J Biol Chem (2015), supra.

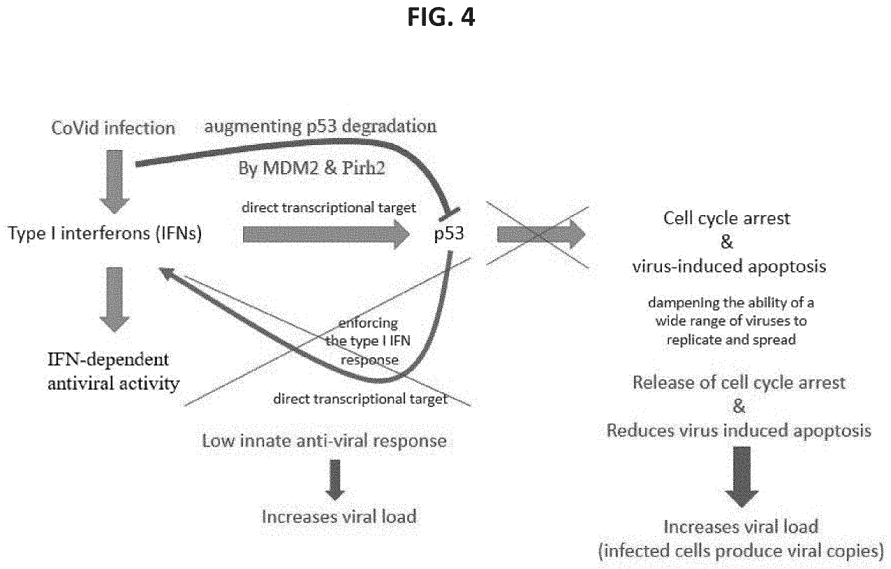

FIG. 5 is a graph illustrating XTT viability assay of human GBM (glioblastoma) LN-18 (p53 mutated) cells. Cell viability 24, 48 and 72 hours following treatment with p53-comprising cell-derived vesicles at different doses is provided as compared to a control (cells grown in the same media but without the exosomes). Of note, a strong response was observed in a dose-depended manner. **t-test p<0.05.

FIG. 6 is a graph illustrating the apoptotic rate of human GBM (glioblastoma) LN-18 (p53 mutated) cells 24 hours following treatment with p53-comprising cell-derived vesicles compared to control (cells grown in the same media but without the exosomes). Depicted by AnnexinV/PI staining.

FIGS. 7A-D are photographs illustrating the specificity of p53 comprising cell-derived vesicles obtained from corneal epithelial cells. Of note, a significant effect is seen in vitro of p53-comprising cell-derived vesicles, as opposed to no effect from administration of a similar composition derived from an adjacent tissue (i.e. from cells in which p53 is present in undetectable levels due to regular MDM2 regulation, which is absent in corneal epithelial cell).

FIG. 8 is a schematic illustration of the in vitro assays carried out as a proof-of-concept illustrating the antiviral efficacy of p53-comprising cell-derived vesicles.

FIG. 9 is a graph illustrating a viability assay. Green circles illustrate the viability of uninfected Vero E6 cells. Blue squares illustrate the viability of SARS-CoV-2 infected Vero E6 cells treated with different doses of p53-comprising cell-derived vesicles obtained from corneal cells.

### DESCRIPTION OF SPECIFIC EMBODIMENTS OF THE INVENTION

The present invention, in some embodiments thereof, relates to cell-derived vesicles comprising wild-type p53 protein and, more particularly, but not exclusively, to the use of same in treatment of viral infections.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Development of anti-viral drugs is imminent to the ability to treat viral infections including the struggle with pandemics. The SARS (severe acute respiratory syndrome) coronavirus 2 (SARS-CoV-2) is a newly discovered member of the family of coronaviruses. It is a respiratory virus that causes a disease known as Covid-19. Symptoms of Covid-19 can range from mild-illness characterized by fever, fatigue, dry cough and shortness of breath, to severe and acute respiratory distress syndrome, renal dysfunction, and multi-organ failure. Currently, there is no specific anti-viral treatment recommended for COVID-19. In patients with severe cases, treatment involves mechanical ventilation and vital organ function support.

While reducing the present invention to practice, the present inventors have uncovered that wild-type p53 provided in cell-derived vesicles to virally infected cells, including SARS-CoV-2 infected cells, can be utilized as an efficient anti-viral therapy. Specifically, the present invention discloses that viral infection can be treated by delivery of wild-type p53 by means of cell-derived vesicles, administered systemically or directly to affected tissues, wherein the wild-type p53 enhances p53 anti-viral functions (e.g. cell cycle arrest and/or apoptosis and reduction of viral load) and enables host resistance against the virus infection.

Thus, according to one aspect of the present invention there is provided a method of treating a viral infection in a subject in need thereof, the method comprising administering to the subject a therapeutically effective amount of cell-derived vesicles comprising wild-type p53, thereby treating the viral infection in the subject.

According to one aspect of the invention, there is provided a therapeutically effective amount of cell-derived vesicles comprising wild-type p53 for use in treating a viral infection in a subject in need thereof.

The term “treating” refers to inhibiting or arresting the development of a pathology (e.g. viral infection) and/or causing the reduction, remission, or regression of a pathology (e.g. viral infection or symptoms associated therewith). Those of skill in the art will understand that various methodologies and assays can be used to assess the development of a pathology, and similarly, various methodologies and assays may be used to assess the reduction, remission or regression of a pathology (e.g. viral infection or symptoms associated therewith, as further discussed below).

The term “treating” also includes preventing the development of a pathology from occurring in a subject who may be at risk for the pathology, but has not yet been diagnosed as having the pathology. It will be appreciated that the treating may be performed alone or in conjunction with other therapies.

The term “viral infection” as used herein refers to the entry of a viral pathogen (i.e. virus) into the body of a host subject. The viral pathogen may be present in cells or tissues of a host subject. Additionally or alternatively, the viral pathogen may be present in bloodstream and/or in other body fluids of a subject (e.g. saliva, semen, pleural, amniotic, pericardial, peritoneal, synovial and cerebrospinal fluids). Viral infection may be accompanied by signs of illness (e.g. fever, cough, etc. as discussed in detail below), but may also be free of such signs. Moreover, viral infection may be accompanied by an inflammatory response including, for example, release of cytokines at the site of infection or a cytokine storm.

As used herein, a “virus” refers to any of group of infectious entities that cannot grow or replicate without a host cell. Viruses typically contain a protein coat and RNA or DNA as of genetic material, they have no semipermeable membrane, and are capable of growth and multiplication only in living cells.

In some embodiments, the viral infection is caused by a DNA virus.

In some embodiments, the viral infection is caused by a RNA virus.

In some embodiments, the viral infection is caused by an enveloped DNA virus.

In some embodiments, the viral infection is caused by an enveloped RNA virus.

Exemplary viruses which can cause the viral infection in accordance with some embodiments of the invention are listed in Table 1, below.

TABLE 1
List of viruses

| Family Name | Representative Viruses |
|---|---|
| Adenoviridae | Human adenovirus types 1 to 57 in seven species (human adenovirus species |
| | A to G) |
| Anelloviridae | Torque teno virus 1 (TTV1), Torque teno mini virus 1, Torque teno midi |
| | virus 1 (type species for numerous viruses in 3 genera) |
| Arenaviridae | Lassa virus, lymphocytic choriomeningitis virus, Junin virus, Machupo virus, |
| | Guanarito virus, Sabia virus, Whitewater Arroyo virus, Chapare virus, Lujo |
| | virus |
| Astroviridae | Human astroviruses (eight serotypes) |
| Bornaviridae | Mammalian 1 bornavirus (formerly Borna disease virus [BDV]) |
| Bunyaviridae | California encephalitis virus, Sin Nombre virus, La Crosse virus, Hantaan |
| | virus, Muerto Canyon virus, Crimean-Congo hemorrhagic fever virus, |
| | Sandfly fever viruses, Rift Valley fever virus, Heartland virus |
| Caliciviridae | Noroviruses, sapoviruses |
| Coronaviridae | SARS-Cov-2 coronavirus, SARS coronavirus; MERS coronavirus; human |
| | coronaviruses OC43, 229E, NL63, and HKU1; human torovirus and other |
| | human enteric coronaviruses |
| Filoviridae | Ebola viruses, Marburg virus |
| Flaviviridae | Genus Alphavirus: dengue virus, yellow fever virus, Japanese encephalitis |
| | virus, West Nile virus, Murray Valley encephalitis virus, Kyasanur |
| | encephalitis virus, tick-borne encephalitis virus, Zika virus |
| | Genus Hepacivirus: hepatitis C virus (HCV) |
| | Genus Pegivirus: GB virus-C (GBV-C) (formerly hepatitis G virus [HGV]) |
| Hepadnaviridae | Hepatitis B virus (HBV) |
| Hepeviridae | Hepatitis E virus (HEV) |
| Herpesviridae | Herpes simplex virus type 1, herpes simplex virus type 2, varicella-zoster |
| | virus, cytomegalovirus, Epstein-Barr virus, human herpesvirus 6, human |
| | herpesvirus 7, human herpesvirus 8 (i.e., Kaposi sarcoma-associated |
| | herpesvirus), herpes simian B virus |
| Orthomyxoviridae | Influenza A virus (e.g. subtype H1N1), influenza B virus, influenza C virus, |
| | Thogoto virus, Dhori virus, Bourbon virus |
| Papillomaviridae | Human papilloma virus (>150 types with various degrees of oncogenicity) |
| Paramyxoviridae | Measles (rubeola) virus, mumps virus, parainfluenza viruses, Hendra virus, |
| | Nipah virus, Menangle virus |
| Parvoviridae | Human parvovirus B19, human bocavirus, adeno-associated viruses |
| Picobirnaviridae | Human picobirnavirus |
| Picornaviridae | Genus Enterovirus: human rhinoviruses (>100 serotypes), enteroviruses |
| | (>100 serotypes, including poliovirus 1-3, coxsackievirus A and B, |
| | echoviruses, and other human enteroviruses) |
| | Genus Hepatovirus: hepatitis A virus (HAV) |
| | Genus Parechovirus: human parecho viruses |
| | Genus Kobuvirus: Aichi virus |
| | Genus Cosavirus: human cosaviruses |
| | Genus Cardiovirus: Vilyuisk human encephalomyelitis virus, Saffold viruses |
| | Genus Salivirus: human klassevirus, salivirus A |
| | Genus Senecavirus: Seneca Valley virusf |
| | Unassigned: Syr-Darya Valley fever virus |
| Pneumoviridae | Respiratory syncytial virus, human metapneumoviruses |
| Polyoma viridae | JC virus, BK virus, KI virus, WU virus, Merkel cell polyomavirus, |
| | lymphotropic polyomavirus, human polyomavirus 6, human polyomavirus 7, |
| | trichodysplasia spinulosa-associated polyomavirus, human polyomavirus |
| Poxviridae | Molluscum contagiosum virus, variola (smallpox) virus, monkeypox virus, |
| | vaccinia virus, orf virus, pseudocowpox virus, Tanapox virus, Yaba monkey |
| | tumor virus |
| Reoviridae | Human rotavirus, Colorado tick fever virus, human reovirus, c Kemerovo |
| | virus |
| Retroviridae | Human immunodeficiency viruses types 1 and 2, human T-lymphocyte |
| | lymphotropic viruses, xenotropic murine leukemia virus-related virus, human |
| | endogenous retroviruses (HERVs), simian foamy virus |
| Rhabdoviridae | Rabies virus, vesicular stomatitis virus, Australian bat lyssavirus, Duvenhage |
| | virus, Mokola virus |
| Togaviridae | Rubella virus; Chikungunya virus; eastern equine, western equine, and |
| | Venezuelan equine encephalitis viruses; Ross River, Sindbis, and Semliki |
| | Forest viruses |
| Delta | Hepatitis delta viruse (HDV) |

In some embodiments, the viral infection is caused by an oncogenic virus (i.e. a virus that causes cancer in humans). Exemplary oncogenic viruses include, but are not limited to, hepatitis B virus (HBV), hepatitis C virus (HCV), human papillomavirus (HPV), Epstein Barr virus (EBV), human herpes virus 8 (HHV8), Merkel cell polyomavirus (MCPyV), Human T-cell leukemia virus type 1 (HTLV-1) and Rous sarcoma virus (RSV).

In some embodiments, the viral infection is caused by a non-oncogenic virus (i.e. a virus that does not cause cancer in humans). Exemplary non-oncogenic viruses include, but are not limited to, Coronavirus (including, but not limited to, SARS coronavirus), Influenza virus, infectious bronchitis virus (IBV), Human immunodeficiency virus (HIV) and Respiratory syncytial virus.

According to one embodiment, viruses that cause the viral infection according to some embodiments of the invention include, but are not limited to, Adenoviruses, Bocaviruses, Coronaviruses (including, but not limited to, SARS coronavirus), Coxsackieviruses, Cytomegalovirus (CMV), Dengue fever virus, Ebola virus, Echovirus, Enterovirus, Epstein-Barr virus, Human Immunodeficiency Virus (HIV, including, but not limited to, HIV-1 and HIV-2), Hantavirus, Human papilloma virus (HPV), Herpes simplex virus (including, but not limited to, HSV-1 and HSV-2), Hepatotropic viruses (including but not limited to Hepatitis A, Hepatitis B, Hepatitis C, Hepatitis D, and Hepatitis E), Influenza virus, infectious bronchitis virus (IBV), Japanese encephalitis virus, Marburg virus, Metapneumovirus, Mumps virus, Norovirus, Poxvirus, Parvovirus, Parainfluenza virus, Papilloma virus, Poliovirus, Retrovirus, Rabies virus, Respiratory syncytial virus, Rotavirus, Rhinovirus, Rous sarcoma virus (RSV), Human T-cell leukemia virus (including, but not limited to, type 1 (HTLV-1) and type 2 (HTLV-2)), Seneca Valley Virus, Semliki forest virus, Smallpox virus, Vaccinia virus, Variola virus, Varicella zoster virus, Vesicular stomatitis virus (VSV), West Nile virus, Yellow fever virus, or a Zika virus.

According to one embodiment, the viral infection is caused by a Human papilloma virus (HPV).

According to one embodiment, the viral infection is caused by a Coronavirus.

As used herein “Coronavirus” refers to enveloped single-stranded RNA viruses that belong to the family Coronaviridae and the order Nidovirales.

Coronaviruses include, but are not limited to, the human coronavirus (HCoV, which typically cause common cold including e.g. HCoV-229E, HCoV-OC43, HCoV-NL63, HCoV-HKU1), transmissible gastroenteritis virus (TGEV), murine hepatitis virus (MHV), bovine coronavirus (BCV), feline infectious peritonitis virus (FIPV), severe acute respiratory syndrome coronavirus (SARS-CoV), Middle East respiratory syndrome coronavirus (MERS-CoV) or severe acute respiratory syndrome coronavirus 2 (SARS-CoV-2).

According to a specific embodiment, the human coronavirus is SARS-CoV-2 (i.e. causing Covid-19 disease).

According to a specific embodiment, the human coronavirus is SARS-CoV.

Methods of determining the presence of a viral infection in a subject are well known in the art and are either based on serology, protein markers, electron microscopy or nucleic acid assays including, but not limited to, PCR and sequencing.

As used herein, the terms “subject” or “subject in need thereof” include animals, preferably mammals, including human beings, at any age or of any gender which may suffer from a viral infection. The subject may be a healthy subject or a subject at any stage of a viral infection, e.g. a subject being asymptomatic for the viral infection, a subject showing preliminary signs of a viral infection, a subject being in a symptomatic stage of the viral infection, or a subject after the symptomatic stage of the viral infection.

According to one embodiment, the subject is afflicted with the viral infection, yet does not necessarily show symptoms of the viral infection (i.e. is an asymptomatic carrier). The subject may be contagious or not contagious.

Symptoms of a viral infection include, for example, fever, cough, sputum production, sore throat, runny or stuffy nose, congestion, sneezing, muscle aches, headaches, dizziness, nausea, diarrhea, fatigue, and malaise.

Symptoms associated with Coronavirus infection (e.g. with SARS-CoV-2) include, for example, fever, chills (with or without repeated shaking), cough, fatigue, runny or stuffy nose, sore throat, nausea, loss of smell and/or taste, shortness of breath, inflammation in the lung, alveolar damage, diarrhea, organ failure, pneumonia and/or septic shock.

According to one embodiment, the symptoms may be present during the primary viral infection. According to one embodiment, the symptoms may persist for a prolonged period of time, e.g. for several weeks or months following the viral infection (i.e. secondary effects of the viral infection).

According to a specific embodiment, when the viral infection is caused by Coronavirus (e.g. SARS-CoV-2), the secondary effects of infection include, but are not limited to, fatigue, shortness of breath, cough, joint pain, muscle pain, chest pain, depression, heart palpitations and pulmonary fibrosis.

According to a specific embodiment, the subject is selected as being high risk for the viral infection (e.g. for Coronavirus e.g. for SARS-CoV-2) or for complications associated therewith (e.g. for pulmonary fibrosis) prior to treatment (e.g. a diabetes subject, an immunocompromised subject, a subject suffering from a lung condition such as e.g. COPD, a subject suffering from a heart condition, a cancer patient, etc.).

According to a specific embodiment, the subject is selected as being positive for the viral infection (e.g. for Coronavirus e.g. for SARS-CoV-2) prior to treatment.

Any method known in the art for detection of a viral infection can be used according to the present teachings including e.g. physical examination, blood tests, serology test, protein markers or nucleic acid assays including, but not limited to, PCR and sequencing.

As mentioned, the subject is treated with a therapeutically effective amount of cell-derived vesicles comprising wild-type p53.

The term “cell-derived vesicles” as used herein refers to externally released vesicles that are obtainable from a cell in any form.

The cell-derived vesicles of the invention have cytoplasmic content which comprises p53 and is entrapped in a cell membrane. The cell-derived vesicles of the invention include membrane markers of the cell.

According to one embodiment, the cell-derived vesicles are generated by disruption of cell membranes using synthetic means, e.g., sonication, homogenization extrusion, etc.

According to one embodiment, the cell-derived vesicles are cell-secreted vesicles.

According to one embodiment, the cell-derived vesicles include, for example, microvesicles (e.g. vesicles shed/bud/bleb from the plasma membrane of a cell and have irregular shapes), membrane particles (e.g. vesicles that shed/bud/bleb from the plasma membrane of a cell and are round-shaped), membrane vesicles (e.g. micro vesicles), exosomes (e.g. vesicles derived from the endo-lysosomal pathway), apoptotic bodies (e.g. vesicles obtained from apoptotic cells).

For example, exosomes are formed by invagination and budding from the limiting membrane of late endosomes. They accumulate in cytosolic multivesicular bodies (MVBs) from where they are released by fusion with the plasma membrane. Alternatively, vesicles similar to exosomes (though somewhat larger, often called ‘microvesicles’ or ‘membrane particles’) can be released directly from the plasma membrane.

The size of cell-derived particles can vary considerably, but typically cell-derived particles have a diameter below 1000 nm.

Cell-derived vesicles (e.g. cell-secreted vesicles) typically have a particle size (e.g. diameter) of about 30, 40, 50, 60, 70, 80, 90, 100, 150, 200, 250 300, 400 or 500 nm.

According to one embodiment, the cell-derived vesicles (e.g. cell-secreted vesicles) have a particle size (e.g. diameter) of about 10-1000 nm, about 10-750 nm, about 10-500 nm, about 10-250 nm, about 10-100 nm, about 10-50 nm, about 10-25 nm, about 10-20 nm, about 20-1000 nm, about 20-750 nm, about 20-500 nm, about 20-250 nm, about 20-100 nm, about 20-50 nm, about 50-1000 nm, about 50-750 nm, about 50-500 nm, about 50-100 nm, about 100-1000 nm, about 100-750 nm, about 100-500 nm, about 100-250 nm, about 150-200, about 200-1000 nm, about 200-750 nm, about 200-500 nm, or about 200-250 nm.

According to one embodiment, the cell-derived vesicles (e.g. cell-secreted vesicles) have a particle size (e.g. diameter) of no more than about 1000 nm, 750 nm, 500 nm, 300 nm, 250 nm, 200 nm, 150 nm, 100 nm, 50 nm, 25 nm, 20 nm or 10 nm.

According to one embodiment, the cell-derived vesicles (e.g. cell-secreted vesicles) have a particle size (e.g. diameter) of about 100-300 nm (e.g. about 150-250 nm).

According to one embodiment, the cell-derived vesicles (e.g. cell-secreted vesicles) have a particle size (e.g. diameter) of about 20-200 nm (e.g. about 30-100 nm).

According to one embodiment, the cell-derived vesicles have an average vesicle size, namely the numbers provided herein relate to discrete vesicles or a vesicle population in which the average vesicle size (e.g. diameter) is of about 30-200 nm (e.g., about 30-180 nm, about 30-100 nm, about 80-220, about 100-200 nm, about 150-200 nm).

According to a specific embodiment, the cell-derived vesicles comprise exosomes.

According to one embodiment, the cell-derived vesicles comprise exosomes having a vesicle size (e.g., diameter) of about 30-250 nm (e.g., about 30-200 nm, e.g. 30-100 nm).

According to a specific embodiment, the cell-derived vesicles comprise microvesicles.

According to one embodiment, the cell-derived vesicles comprise microvesicles having a vesicle size (e.g., diameter) of about 10-1000 nm (e.g., about 50-300, e.g. about 150-250 nm).

According to one embodiment, the cell-derived vesicles are native cell-derived vesicles, e.g. are obtained from natural cells or obtained from their natural environment (as discussed below).

According to one embodiment, the cell-derived vesicles are not artificial cell-derived vesicles (e.g. coated liposomes).

Depending on the cellular origin, cell-derived vesicles harbor biological material including e.g. nucleic acids (e.g. RNA or DNA), or cytoplasmic content including proteins, peptides, polypeptides, antigens, lipids, carbohydrates, and proteoglycans. For example, various cellular proteins can be found in cell-derived vesicles including MHC molecules, tetraspanins, adhesion molecules and metalloproteinases.

According to one embodiment, the cell-derived vesicles comprise the membrane arrangement of a cell. They may comprise any cell-originated molecules, carbohydrates and/or lipids that are typically presented in a cell membrane. Furthermore, each type of cell-derived vesicles express distinctive biomarkers. For example, membrane particles typically express CD133 (prominin-1), microvesicles typically express integrins, selectins, and CD40, while exosomes typically express CD63, CD81, CD9, CD82, CD37, CD53, or Rab-5b.

Cell-derived vesicles can be identified using methods well known in the art, e.g. by electron microscopy (EM) and nanoparticle tracing analysis (NTA), and their biomarker expression can be determined using methods well known in the art, for example, by Western blot, ELISA and Flow cytometry assay (e.g. FACS).

According to one embodiment, cell-derived particles are obtained from cells of a human or animal tissue which naturally express high levels of p53.

According to one embodiment, cell-derived particles are obtained from cells of a human or animal tissue which endogenously express p53.

The term “endogenous” as used herein refers to any polynucleotide or polypeptide which is naturally expressed within the cells from which the cell-derived vesicles are obtained.

As used herein, the phrase “exogenous” refers to a polynucleotide or polypeptide which may not be naturally expressed within the cells from which the cell-derived vesicles are obtained.

As used herein the terms “p53” or “p53 protein” refer to the tumor suppressor protein p53 (also referred to Tumor Protein P53 or TP53, Cellular tumor antigen p53, Antigen NY-CO-13, Phosphoprotein p53). p53 generally functions as a nuclear protein (transcription factor) that plays an essential role in the regulation of cell cycle, apoptosis and senescence. Thus, p53 is a DNA-binding protein containing DNA-binding, oligomerization and transcription activation domains. It is postulated to bind as a tetramer to a p53-binding site and activate expression of downstream genes that inhibit growth and/or invasion, and hence acting, in its wild-type form, as a tumor suppressor, and mediator of virus-induced cell cycle arrest, apoptosis and other innate immune responses (e.g. transactivation of the expression of innate immunity-related genes e.g. IRF9, TRL3, ISG15, and MCP-1).

The term “wild-type” as used herein refers to a p53 which has not been modified or altered. Thus, the wild-type p53 of some embodiments of the invention is not a mutated p53 protein, i.e. is a p53 protein performing its innate anti-viral defense function.

According to one embodiment, the p53 protein is a human p53.

Exemplary human p53 proteins include, but are not limited to, those listed under GenBank accession nos. NP_000537.3, NP_001119584.1, NP_001119585.1, NP_001119586.1, NP_001119587.1, NP_001119588.1 and NP_001119589.1.

According to one embodiment, the p53 protein is an animal p53 protein (e.g. mammal, fish, bird, reptile, amphibian, insect, such as of a farm animal, e.g. cattle, sheep, goat, chicken, pig, horse; mouse; elephant, as further discussed below).

According to one embodiment, the p53 protein is a mammalian p53 protein.

According to one embodiment, the p53 protein is a swine (Sus scrofa) p53 protein. Exemplary swine p53 proteins include, but are not limited to, those listed under GenBank accession no. NP_998989.3.

According to one embodiment, the p53 protein is a cattle (Bos TAURUS) p53 protein. Exemplary cattle p53 proteins include, but are not limited to, those listed under GenBank accession no. NP_776626.1.

According to one embodiment, the p53 protein is a sheep (Ovis aries) p53 protein. Exemplary sheep p53 proteins include, but are not limited to, those listed under GenBank accession nos. XP_011954275.1, XP_011954277.1, XP_004017979.1 and XP_011954276.1. According to one embodiment, the p53 protein is of an elephant (Loxodonta africana) p53 protein. Exemplary elephant p53 proteins include, but are not limited to, those listed under GenBank accession nos. G3UI57, G3UJ00, G3UK14, G3UHY3, G3TS21, G3U6D1, G3T035, G3U6U6, G3UDE4, G3ULT4, G3UAZ0 and G3UHE5.

According to one embodiment, the p53 protein is of a goat p53 protein.

According to one embodiment, the p53 protein is of a rabbit p53 protein.

According to one embodiment, the p53 protein is a mouse (Mus musculus) p53 protein. Exemplary mouse p53 proteins include, but are not limited to, those listed under GenBank accession nos. NP_001120705.1 and NP_035770.2.

According to one embodiment, the p53 protein is a bird p53 protein.

According to one embodiment, the p53 protein is a chicken (Gallus gallus) p53 protein. Exemplary chicken p53 proteins include, but are not limited to, those listed under GenBank accession no. NP_990595.1.

According to one embodiment, the p53 protein is a fish, reptile, amphibian, insect or arachnid p53 protein.

According to one embodiment, the wild-type p53 protein comprises an active wild-type p53 protein.

According to one embodiment, the active wild-type p53 protein comprises a phosphorylated wild-type p53 protein.

According to one embodiment, phosphorylation of p53 is at the N- and/or C-terminal domain of p53. For example, p53 can be phosphorylated at serine (e.g. serine 15, 33, 37 or 392) or threonine (e.g. threonine 18) residues within the N- and/or C-terminal regions of the protein. Phosphorylation can be detected by any method known in the art, such as by Western Blot analysis.

According to one embodiment, phosphorylation of p53 stabilizes and/or activates and/or prolongs the half-life and/or increases the levels of p53 protein in a cell. Thus, for example, phosphorylation of p53 prolongs the half-life of p53 from several minutes (e.g. from about 1, 2, 5, 10, 20, 30, 40, 50 or 60 minutes) to several hours (e.g. to about 0.5, 1, 2, 3, 5, 10, 15, 20, 25, 30, 40, 50 or 60 hours). According to one embodiment, phosphorylation of p53 prolongs the half-life of p53 by several-fold, such as by about 2, 3, 4, 5, 6, 7, 8, 9 or 10 times.

According to some embodiments of the invention, treatment of a cell with a DNA damaging agent phosphorylates p53. DNA damaging agents are discussed in detail below.

Determining that a p53 protein is active can be carried out using any method known in the art, such as but not limited to, Enzyme linked immunosorbent assay (ELISA), Western blot, Radio-immunoassay (RIA), Fluorescence activated cell sorting (FACS), Immunohistochemical analysis, In situ activity assay and In vitro activity assays.

According to one embodiment, the cell-derived vesicles contain at least about 0.0001%, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% or more endogenous wild-type p53 protein (i.e., p53 protein not added exogenously i.e., resulting from gene expression in the cell source) of the total vesicular content.

According to a specific embodiment, the cell-derived vesicles contain an amount of at least 0.0001% endogenous wild-type p53 protein of the total vesicular content.

According to a specific embodiment, the cell-derived vesicles contain an amount of at least 0.001% endogenous wild-type p53 protein of the total vesicular content.

According to a specific embodiment, the cell-derived vesicles contain an amount of at least 0.01% endogenous wild-type p53 protein of the total vesicular content.

According to one embodiment, the cell-derived vesicles are obtained from cells which do not naturally express MDM2 polypeptide.

As used herein the term “MDM2” or “MDM2 polypeptide” refers to the Mouse Double Minute 2, Human Homolog Of. MDM2 generally functions as a p53-binding protein which negatively regulates p53. Accordingly, under normal conditions, MDM2 maintains low intracellular levels of p53 by targeting p53 to the proteasome for rapid degradation and inhibits p53's transcriptional activity.

According to one embodiment, the MDM2 polypeptide is a human MDM2 polypeptide. Exemplary human MDM2 polypeptides include, but are not limited to, those listed under GenBank accession nos. NP_001138809.1, NP_001138811.1, NP_001138812.1, NP_001265391.1 and NP_002383.2.

According to one embodiment, the MDM2 polypeptide is an animal MDM2 polypeptide (e.g. a mammal, fish, bird, reptile, amphibian, insect, such as of a farm animal, e.g. cattle, sheep, goat, chicken, pig, horse; mouse; elephant, as further discussed below). Exemplary MDM2 polypeptides are set forth in GenBank Accession no. Q9PVL2-1 for Gallus gallus (Chicken), GenBank Accession no. NP_001092577.1 for Bos taurus (Cattle), GenBank Accession no. W5PWI5-1 for Ovis aries (sheep) and GenBank Accession no. NP_001098773.1 for Sus scrofa (swine).

According to one embodiment, the cell-derived vesicles contain endogenous MDM2 polypeptide at a level not exceeding 0.001%, 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% of the total vesicular content.

According to a specific embodiment, the cell-derived vesicles contain endogenous MDM2 polypeptide at a level not exceeding 0.1% of the total vesicular content.

According to a specific embodiment, the cell-derived vesicles contain endogenous MDM2 polypeptide at a level not exceeding 0.5% of the total vesicular content.

Without being bound by theory, the low levels of MDM2 in a cell (and in a cell-derived vesicle derived therefrom, e.g. a level not exceeding 0.5% of the total vesicular content), such as in corneal epithelial cells, enable the naturally high p53 expression, as MDM2 is a negative regulator of p53.

According to one embodiment, the cell-derived vesicles contain additional peptides, polypeptides and proteins such as tumor suppressors, immune modulators, MHC molecules, cytoskeletal proteins, membrane transport and fusion proteins, tetraspanins and/or proteins belonging to the heat-shock family, non-coding RNA molecules (e.g. miRNA, siRNAs, piRNAs, snoRNAs, snRNAs, exRNAs, scaRNAs, tRNAs, rRNAs and long ncRNAs). Without being bound by theory, these factors are typically cellular components found in the cell cytoplasm and are incorporated into the cell-derived vesicles upon their production (e.g. by shedding/budding/blebbing).

Exemplary tumor suppressors include, but are not limited to, Retinoblastoma protein (pRb), maspin, pVHL, APC, CD95, STS, YPEL3, ST7, ST14, BRMS1, CRSP3, DRG1, KAI1, KISS1, NM23 and TIMPs.

Exemplary immune modulators include, but are not limited to, Hsp70 and galectin-5.

Exemplary miRNAs include, but are not limited to, miR-29b, miR-34b/c, miR-126, miR-150, miR-155, miR-181a/b, miR-375, miR-494, miR-495 and miR-551a.

Additional factors which may be found in cell-derived vesicles, include but are not limited to, those discussed in M. Shen et al., Biochimica et Biophysica Acta 1864 (2016) 787-793; Botling Taube A, et al., Br J Ophthalmol (2019) 103:1190-1194; Poe et al., Cells (2020) 9: 2175; and Dyrlund et al., J. Proteome Res. (2012) 11: 4231-4239, all incorporated herein by reference.

According to one embodiment, the additional peptides, polypeptides (e.g. immune modulators) or non-coding RNA molecules are endogenous to the cells from which the cell-derived vesicles are derived (e.g. originating from the cells releasing the cell-derived vesicles).

According to one embodiment, the cell-derived vesicles comprise components (e.g. peptides, polypeptides or non-coding RNA molecules) which are not native to the cells from which the cell-derived vesicles are derived (as further discussed below).

According to one embodiment, the cell-derived vesicles are obtained from natural cells.

As mentioned, the cell-derived vesicles may be obtained from cells which naturally express p53.

According to one embodiment of the invention, the cell-derived vesicles are obtained from cells which are not genetically manipulated to express p53 proteins or recombinant versions thereof (e.g. non-genetically modified cells).

According to one embodiment of the invention, the cell-derived vesicles are obtained from cells which are genetically manipulated to express p53 proteins or recombinant versions thereof, e.g. to express higher levels of p53 protein in cells naturally expressing p53 (e.g. genetically modified cells).

According to one embodiment, the cell-derived vesicles are obtained from cells which express at least about 0.0001%, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% or more endogenous wild-type p53 protein of the total cellular proteins. Methods of measuring expression of p53 proteins in a cell are well known in the art and include, e.g. ELISA, Western blot analysis, and Flow cytometry assay (e.g. FACS).

According to a specific embodiment, the cell-derived vesicles are obtained from cells which express at least about 0.0001% endogenous wild-type p53 protein of the total cellular proteins.

According to a specific embodiment, the cell-derived vesicles are obtained from cells which express at least about 0.001% endogenous wild-type p53 protein of the total cellular proteins.

According to a specific embodiment, the cell-derived vesicles are obtained from cells which express at least about 0.01% endogenous wild-type p53 protein of the total cellular proteins.

According to a specific embodiment, the cell-derived vesicles are obtained from cells which express at least about 0.1% endogenous wild-type p53 protein of the total cellular proteins.

According to a specific embodiment, the cell-derived vesicles are obtained from cells which express at least about 0.5% endogenous wild-type p53 protein of the total cellular proteins.

According to one embodiment, the cell-derived vesicles are obtained from cells which do not naturally express endogenous MDM2 polypeptide.

According to one embodiment, the cell-derived vesicles are obtained from cells which express endogenous MDM2 polypeptide at a level not exceeding 0.001%, 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10% of the total cellular proteins. Methods of measuring expression of MDM2 polypeptides in a cell are well known in the art and include, e.g. ELISA, Western blot analysis, and Flow cytometry assay (e.g. FACS).

According to a specific embodiment, the cell-derived vesicles are obtained from cells which express endogenous MDM2 polypeptide at a level not exceeding 0.1% of the total cellular proteins.

According to a specific embodiment, the cell-derived vesicles are obtained from cells which express endogenous MDM2 polypeptide at a level not exceeding 0.5% of the total cellular proteins.

According to one embodiment, the cell-derived vesicles are obtained from cells which have been treated with a MDM2 inhibitor. MDM2 inhibitors are well known in the art and include, for example, Nutlin-3, Spirooxindoles and 1,4-benzodiazepine-2,5-diones (BDP), as discussed in detail in Khoury and Domling, Curr Pharm Des. (2012) 18(30): 4668-4678, incorporated herein by reference.

According to one embodiment, cell-derived vesicles (i.e. comprising a wild-type p53) are obtained from healthy cells (e.g. non-cancerous cells).

According to one embodiment, cell-derived vesicles (i.e. comprising a wild-type p53) are obtained from genetically non-modified cells.

According to one embodiment, cell-derived vesicles (i.e. comprising a wild-type p53) are obtained from human cells.

According to one embodiment, cell-derived vesicles (i.e. comprising a wild-type p53) are obtained from animal cells.

According to one embodiment, cell-derived vesicles (i.e. comprising a wild-type p53) are obtained from cells of an animal selected from a fish, an amphibian, an insect, a reptile, an arachnid, a bird and a mammal.

According to one embodiment, the animal is a mammal, including but not limited to a mouse, a rat, a hamster, a guinea pig, a gerbil, a hamster, a rabbit, a cat, a dog, a pig (e.g. swine), a cattle (e.g. a cow or a bull), a goat, a sheep, a primate, an elephant, a deer, an elk, and a horse.

According to one embodiment, the animal is a bird, including but not limited to, a chicken, a turkey, a duck, a goose, a swan and a ground tit.

According to one embodiment, the animal is a fish, a reptile (e.g. lizard or snake), an amphibian (e.g. a frog, a toad or a tadpole), an insect and an arachnid. According to one embodiment, cell-derived vesicles comprising a wild-type p53 (e.g. active wild-type p53) are obtained from cells of various tissues including, but not limited to, eye tissues (e.g. corneal epithelium tissue), epidermis (e.g. skin epidermis), testicles, epithelium of small intestines and a brain tissues (e.g. cerebellum, hippocampus, hypothalamus, pons, thalamus and upper cerebral spine).

According to a specific embodiment, cell-derived vesicles comprising a wild-type p53 (e.g. active wild-type p53) are obtained from cells of an eye tissue (e.g., of human, pig, cattle or a chicken).

According to a specific embodiment, cell-derived vesicles comprising a wild-type p53 (e.g. active wild-type p53) are obtained from epidermal cells (e.g. of a skin tissue) of amphibians including frog or toad, or of lip tissue (e.g. around the mouth) for tadpoles.

According to one embodiment, cell-derived vesicles comprising a wild-type p53 (e.g. active wild-type p53) are obtained from various cell types, including but not limited to, eye cells (e.g. corneal epithelium cells), intestinal epithelial cells, brain hippocampus cells and other cell types.

According to one embodiment, cell-derived vesicles are obtained from eye cells.

Eye cells refer to any cell existing in an eye, including cells existing in eyelid, conjunctiva and cornea.

Accordingly, cell-derived vesicles comprising a wild-type p53 (e.g. active wild-type p53) may be obtained from any eye cells including but not limited to, cells of the cornea tissue (e.g. epithelial cells, stem cells etc.), cells of melanocytes.

According to a specific embodiment, eye cells from which cell-derived vesicles comprising a wild-type p53 (e.g. active wild-type p53) can be obtained include corneal cells. In humans, the cornea is stated to be composed of five layers from the external side (body surface) in order, and is composed of corneal epithelium, Bowman's membrane (external boundary line), Lamina propria, Descemet's membrane (internal boundary line), and corneal endothelium from the external side.

Exemplary corneal cells from which cell-derived vesicles comprising a wild-type p53 (e.g. active wild-type p53) can be obtained, include but are not limited to, corneal epithelial cells.

According to a specific embodiment, eye cells from which cell-derived vesicles comprising a wild-type p53 (e.g. active wild-type p53) can be obtained include corneal epithelial stem cells.

According to a specific embodiment, cell-derived vesicles comprising a wild-type p53 (e.g. active wild-type p53) can be obtained from testicular cells. In humans, the testes typically contains germ cells (that differentiate into mature spermatozoa), Sertoli cells (germ-cell-supporting cells), Peritubular myoid cells (which surround the seminiferous tubules) and testosterone-producing cells called Leydig (interstitial) cells.

According to one embodiment, cell-derived vesicles are not obtained from blood cells, e.g. T cells, B cells, mononuclear cells.

Depending on the application, the cell-derived vesicles comprising a wild-type p53 may be obtained from cells of an organism which is syngeneic or non-syngeneic with a subject to be treated (discussed in detail herein below).

As used herein, the term “syngeneic” cells refer to cells which are essentially genetically identical with the subject or essentially all lymphocytes of the subject. Examples of syngeneic cells include cells derived from the subject (also referred to in the art as an “autologous”), from a clone of the subject, or from an identical twin of the subject.

As used herein, the term “non-syngeneic” cells refer to cells which are not essentially genetically identical with the subject or essentially all lymphocytes of the subject, such as allogeneic cells or xenogeneic cells.

As used herein, the term “allogeneic” refers to cells which are derived from a donor who is of the same species as the subject, but which is substantially non-clonal with the subject. Typically, outbred, non-zygotic twin mammals of the same species are allogeneic with each other. It will be appreciated that an allogeneic cell may be HLA identical, partially HLA identical or HLA non-identical (i.e. displaying one or more disparate HLA determinant) with respect to the subject.

As used herein, the term “xenogeneic” refers to a cell which substantially expresses antigens of a different species relative to the species of a substantial proportion of the lymphocytes of the subject. Typically, outbred mammals of different species are xenogeneic with each other.

The present invention envisages that xenogeneic cells are derived from a variety of species. Thus, according to one embodiment, the cell-derived vesicles may be obtained from cells of any animal (e.g. mammal). Suitable species origins for the cell-derived vesicles (or cells releasing same) comprise the major domesticated or livestock animals and primates. Such animals include, but are not limited to, poultry (e.g. chicken), porcines (e.g. pig or swine), bovines (e.g., cow), equines (e.g., horse), ovines (e.g., goat, sheep), felines (e.g., Felis domestica), canines (e.g., Canis domestica), rodents (e.g., mouse, rat, rabbit, guinea pig, gerbil, hamster), primates (e.g., chimpanzee, rhesus monkey, macaque monkey, marmoset), and elephants.

Cell-derived vesicles (or cells releasing same) of xenogeneic origin (e.g. porcine origin) are preferably obtained from a source which is known to be free of zoonoses, such as porcine endogenous retroviruses. Similarly, human-derived cell-derived vesicles, cells or tissues are preferably obtained from substantially pathogen-free sources.

According to one embodiment, the cell-derived vesicles of the invention are obtained from cells allogeneic with the subject.

According to one embodiment, the cell-derived vesicles of the invention are obtained from cells xenogeneic with the subject.

According to one embodiment, the cell-derived vesicles of the invention are obtained from cells syngeneic with the subject (e.g. autologous).

Depending on the application and available sources, the cell-derived vesicles of the invention are obtained from cells of a prenatal organism, postnatal organism, an adult or a cadaver. Such determinations are well within the ability of one of ordinary skill in the art.

Obtaining cell-derived vesicles may be carried out using any method known in the art. For example, cell-derived vesicles can be isolated (i.e. at least partially separated from the natural environment e.g., from a body) from any biological sample (e.g., fluid or hard tissue) comprising cell-derived vesicles. Examples of fluid samples include, but are not limited to, whole blood, plasma, serum, spinal fluid, lymph fluid, bone marrow suspension, cerebrospinal fluid, brain fluid, ascites (e.g. malignant ascites), tears, saliva, sweat, urine, semen, sputum, ear flow, vaginal flow, secretions of the respiratory, intestinal and genitourinary tracts, milk, amniotic fluid, and samples of ex vivo cell culture constituents. Examples of tissue samples include, but are not limited to, surgical samples, biopsy samples, tissues, feces, and cultured cells. According to a specific embodiment, the tissue sample comprises a whole or partial organ (e.g. eye, brain, testicle, skin, intestine), such as those obtained from a cadaver or from a living subject undergoing whole or partial organ removal.

Methods of obtaining such biological samples are known in the art, and include without being limited to, standard blood retrieval procedures, standard urine and semen retrieval procedures, lumbar puncture, fine needle biopsy, needle biopsy, core needle biopsy and surgical biopsy (e.g., organ or brain biopsy), buccal smear and lavage. Regardless of the procedure employed, once a biopsy/sample is obtained cell-derived vesicles can be obtained therefrom.

According to one embodiment, the biological sample comprises cell-derived vesicles (or is further processed to comprise cell-derived vesicles, such as cell-secreted vesicles, as discussed below) and is essentially without intact cells.

According to a specific embodiment, the biological sample (e.g. processed sample) comprises less than 1%, 2%, 5%, 10%, 20%, 30%, 40%, 50%, 60%, 70%, 80% or 90% intact cells per ml fluid sample.

However, the biological sample may contain some cells or cell contents. The cells can be any cells which are derived from the subject (as discussed in detail above).

The volume of the biological sample used for obtaining cell-derived vesicles can be in the range of between 0.1-1000 mL, such as about 1000, 750, 500, 250, 100, 75, 50, 25, 15, 10, 9, 8, 7, 6, 5, 4, 3, 2, 1 or 0.1 mL.

The biological sample of some embodiments of the invention may comprise cell-derived vesicles in various ranges, e.g. 1, 5, 10, 15, 20, 25, 50, 100, 150, 200, 250, 500, 1000, 2000, 5000, 10,000, 50,000, 100,000, 500,000, 1×10⁶ or more cell-derived vesicles.

According to one embodiment, cell-derived vesicles (e.g. cell-secreted vesicles) are obtained from cell lines or primary cultures of cells expressing at least about 0.0001% endogenous wild-type p53 protein.

According to one embodiment, obtaining cell-derived vesicles from a biological sample is carried out without the use of a DNA damaging agent.

According to one embodiment, in order to increase secretion of cell-derived vesicles from cells (e.g. cell-secreted vesicles), the cells are treated with a DNA damaging agent. Any known DNA damaging agent may be used in accordance with the present teachings as further discussed below.

According to one embodiment, cell-derived vesicles (e.g. cell-secreted vesicles) are obtained from a freshly collected biological sample or from a biological sample that has been stored cryopreserved or cooled.

According to one embodiment, cell-derived vesicles (e.g. cell-secreted vesicles) are obtained from a culture medium in which the cells have been cultured.

For example, cell-derived vesicles (e.g. cell-secreted vesicles, including exosomes) can be isolated from the biological sample by any method known in the art. Suitable methods are taught, for example, in U.S. Pat. Nos. 9,347,087 and 8,278,059, incorporated herein by reference.

For example, cell-derived vesicles (e.g. cell-secreted vesicles, including exosomes) may be obtained from a fluid sample by first removing any debris from the sample e.g. by precipitation with a volume-excluding polymer (e.g. polyethylene glycol (PEG) or dextrans and derivatives such as dextran sulfate, dextran acetate, and hydrophilic polymers such as polyvinyl alcohol, polyvinyl acetate and polyvinyl sulfate). Methods of clarification include centrifugation, ultracentrifugation, filtration or ultrafiltration. The skilled artisan is aware of the fact, that an efficient separation might require several centrifugation steps using different centrifugation procedures, temperatures, speeds, durations, rotors, and the like. For example, suitable volume-excluding polymers may have a molecular weight between 1000 and 1,000,000 daltons. In general, when higher concentrations of cell-derived vesicles (e.g. exosomes) are present in a sample, lower molecular weight polymers may be used. Volume-excluding polymers may be used at a final concentration of from 1% to 90% (w/v) upon mixing with the sample. A variety of buffers commonly used for biological samples may be used for incubation of the cell-derived vesicles (e.g. exosome) sample with the volume-excluding polymer including phosphate, acetate, citrate and TRIS buffers. The pH of the buffer may be any pH that is compatible with the sample, but a typical range is from 6 to 8. Incubation of the biological sample with the volume-excluding polymer may be performed at various temperatures, e.g. 4° C. to room temperature (e.g. 20° C.). The time of incubation of the sample with the volume-excluding polymer may be any, typically in the range 1 minute to 24 hours (e.g. 30 minutes to 12 hours, 30 minutes to 6 hours, 30 minutes to 4 hours, or 30 minutes to 2 hours). One of skill in the art is aware that the incubation time is influenced by, among other factors, the concentration of the volume-excluding polymer, the molecular weight of the volume-excluding polymer, the temperature of incubation and the concentration of cell-derived vesicles (e.g. exosomes) and other components in the sample. After completion of the incubation of the sample with the volume-excluding polymer the precipitated cell-derived vesicles (e.g. exosomes) may be isolated by centrifugation, ultracentrifugation, filtration or ultrafiltration.

According to one embodiment, cell-derived vesicles (e.g. exosomes) are separated from a biological fluid sample by first centrifugation of the biological sample (e.g. fluid sample such as plasma) at 1000×g for 15 minutes, then passing the sample through a filter (e.g. 0.1-0.5 μm filter, e.g. 0.2 μm filter) and centrifugation at about 100,000×g for 60-120 minutes (e.g. 90 minutes). Centrifugation can be repeated (e.g. after suspending the pellet in phosphate-buffered saline (PBS)) under the same conditions.

When isolating cell-derived vesicles from tissue, cell line or primary culture sources it may be necessary to homogenize the tissue in order to obtain a homogenate containing cell-derived vesicles. When isolating cell-derived vesicles from tissue samples it is important to select a homogenization procedure that does not result in disruption of the cell-derived vesicles.

According to one embodiment, cell-derived vesicles are isolated from a tissue (e.g. eye tissue or testicle tissue) by first harvesting the tissue (e.g. eye tissue or testicle tissue) from a donor (e.g. animal or human) and homogenating the tissue as to obtain a homogenate. The entire tissue may be used, or alternatively a specific part of the tissue may be used. The cell-derived vesicles are then isolated by centrifugation, ultracentrifugation, filtration or ultrafiltration.

According to one embodiment, the tissue is kept in ice prior to homogenization thereof.

According to one embodiment, the cell line or primary culture is cultured in a culture medium prior to obtaining a cell-derived vesicles therefrom. One of ordinary skill in the art is capable of determining the length of time of which the cells may be cultured. According to one embodiment, the cells are cultured for 12 hours, 24 hours, 36 hours, 48 hours, 3 days, 4 days, 5 days, 6 days, 7 days, 10 days, 14 days, 21 days, 30 days or more.

According to one embodiment, the sample may be further purified or concentrated prior to use. For example, a heterogeneous population of cell-derived vesicles can be quantitated (i.e. total level of cell-derived vesicles in a sample), or a homogeneous population of cell-derived vesicles, such as a population of cell-derived vesicles with a particular size, with a particular marker profile, obtained from a particular type of biological sample (e.g. urine, serum, plasma, etc.) or derived from a particular cell type (e.g. eye cells, brain cells, skin cells, epithelial cells, intestinal cells) can be isolated from a heterogeneous population of cell-derived vesicles and quantitated.

According to one embodiment, cell-derived vesicles are purified or concentrated from a biological sample using size exclusion chromatography, density gradient centrifugation, differential centrifugation, nanomembrane ultrafiltration, immunosorbent capture, affinity purification, microfluidic separation, or combinations thereof.

Size exclusion chromatography, such as gel permeation columns, centrifugation or density gradient centrifugation, and filtration methods can be used. For example, cell-derived vesicles can be isolated by differential centrifugation, anion exchange and/or gel permeation chromatography (as described e.g. in U.S. Pat. Nos. 6,899,863 and 6,812,023), sucrose density gradients, organelle electrophoresis (as described e.g. in U.S. Pat. No. 7,198,923), magnetic activated cell sorting (MACS), or with a nanomembrane ultrafiltration concentrator. Thus, various combinations of isolation or concentration methods can be used as known to one of skill in the art.

Sub-populations of cell-derived vesicles may be isolated using other properties of the cell-derived vesicles such as the expression of other immune modulators, cytoskeletal proteins, membrane transport and fusion proteins, tetraspanins and/or proteins belonging to the heat-shock family (as discussed in detail hereinabove). Any method known in the art for measuring expression of a protein can be used, such as but not limited to, ELISA, Western blot analysis, FACS, Immunohistochemical analysis, In situ activity assay and In vitro activity assays. Furthermore, the contents of the cell-derived vesicles may be extracted for characterization of cell-derived vesicles containing any of the above mentioned polypeptides (as discussed in detail hereinabove).

According to one embodiment, cell-derived vesicles are selected for expression of activated (e.g. phosphorylated) wild-type p53 (e.g. phosphorylated). Any method known in the art for measuring expression of p53 protein or phosphorylated variant thereof can be used, such as but not limited to, ELISA, Western blot analysis, FACS, Immunohistochemical analysis, In situ activity assay and In vitro activity assays.

According to one embodiment, the contents of the cell-derived vesicles may be extracted for characterization of cell-derived vesicles containing activated wild-type 53.

Additionally or alternatively, sub-populations of cell-derived vesicles may be isolated using other properties of the cell-derived vesicles such as the presence of surface markers. Surface markers which may be used for fraction of cell-derived vesicles include but are not limited to tumor markers, cell type specific markers and MHC class II markers. MHC class II markers which have been associated with cell-derived vesicles include HLA DP, DQ and DR haplotypes. Other surface markers associated with cell-derived vesicles include, but are not limited to, CD9, CD81, CD63, CD82, CD37, CD53, or Rab-5b (Thery et al. Nat. Rev. Immunol. 2 (2002) 569-579; Valadi et al. Nat. Cell. Biol. 9 (2007) 654-659).

As an example, cell-derived vesicles having CD63 on their surface may be isolated using antibody coated magnetic particles e.g. using Dynabeads®, super-paramagnetic polystyrene beads which may be conjugated with anti-human CD63 antibody either directly to the bead surface or via a secondary linker (e.g. anti-mouse IgG). The beads may be between 1 and 4.5 μm in diameter. Accordingly, the antibody coated Dynabeads® may be added to a cell-derived vesicle sample (e.g. prepared as described above) and incubated at e.g. 2-8° C. or at room temperature from 5 minutes to overnight. Dynabeads® with bound cell-derived vesicles may then be collected using a magnet. The isolated, bead bound cell-derived vesicles may then be resuspended in an appropriate buffer such as phosphate buffered saline and used for analysis (qRT-PCR, sequencing, western blot, ELISA, flow cytometry, etc. as discussed below). Similar protocols may be used for any other surface marker for which an antibody or other specific ligand is available. Indirect binding methods such as those using biotin-avidin may also be used.

Determining the level of cell-derived vesicles (e.g. exosomes) in a sample can be performed using any method known in the art, e.g. by ELISA, using commercially available kits such as, for example, the ExoQuick kit (System Biosciences, Mountain View, Calif.), magnetic activated cell sorting (MACS) or by FACS using an antigen or antigens which bind general cell-derived vesicles (e.g. exosome) markers, such as but not limited to, CD63, CD9, CD81, CD82, CD37, CD53, or Rab-5b.

According to one embodiment, the cell-derived vesicles according to the present invention are devoid of intact cells.

As used herein, the phrase “devoid of intact cells”, when relating to the compositions of the present invention relates to a composition that is essentially without intact cells.

According to a specific embodiment, the composition comprises less than 1%, 2%, 3%, 4%, 5%, 10%, 15%, or 20% intact cells per ml fluid sample.

According to one embodiment, the composition of the present invention which is substantially free of intact cells comprises no more than 1 intact cell per about 100 cell-derived vesicles, no more than 1 intact cell per about 1,000 cell-derived vesicles, no more than 1 intact cell per about 10,000 cell-derived vesicles, no more than 1 intact cell per about 100,000 cell-derived vesicles, no more than 1 intact cell per about 1 million cell-derived vesicles, no more than 1 intact cell per about 10 million cell-derived vesicles, no more than 1 intact cell per about 100 million cell-derived vesicles, no more than 1 intact cell per about 1 billion cell-derived vesicles, no more than 1 intact cell per about 10 billion cell-derived vesicles, or essentially does not comprise any intact cells.

Measuring the number of intact cells in a composition can be carried out using any method known in the art, such as by light microscopy or cell staining methods.

According to one embodiment, at least 10%, 20%, 30%, 40%, 50%, 60%, 70%, 80%, 90%, or 100% of the proteins (e.g. wild-type p53) in the preparation are in the cell-derived vesicles.

According to a specific embodiment, at least 50% of the proteins (e.g. wild-type p53) in the preparation are in the cell-derived vesicles.

According to one embodiment, in order to stabilize and/or activate and/or prolong the half-life and/or increase the cellular levels of the p53 protein in a cell-derived vesicles, the wild-type p53 is subjected to phosphorylation.

According to one embodiment, phosphorylation of p53 is performed by exposure to a DNA damaging agent.

As used herein, the term “DNA damaging agent” refers to any agent which causes damage either directly or indirectly to the nucleotides in the genome.

Exemplary DNA damaging agent include, but are not limited to, x-ray, ultraviolet radiation (UV); ionizing radiation (IR) (e.g. gamma irradiation); chemotherapeutic agent; chemical compounds e.g. platinum-based compounds such as cisplatin; intercalating agents e.g. benzo[a]pyrenes, daunorubicin and actinomycin-D; DNA alkylating agents e.g. nitrogen mustards, methyl methanesulphonate (MMS), N-nitroso-N-methylurea (NMU) and N-ethyl-N-nitrosourea (ENU); psoralens; oxidative stress; hypoxia; and nutrient deprivation.

According to a specific embodiment, the DNA damaging agent is a UV irradiation.

According to one embodiment, the tissue is treated with a DNA damaging agent prior to homogenization thereof. According to one embodiment, this step is performed in a donor (e.g. animal or human) prior to harvesting of the tissue. Additionally or alternatively, a tissue is treated with a DNA damaging agent following harvesting thereof from a donor (e.g. animal or human).

According to one embodiment, the cells are treated with a DNA damaging agent prior to isolation of the cell-derived vesicles. According to one embodiment, this step is performed in a tissue culture plate.

According to one embodiment, the isolated cell-derived vesicles are treated with a DNA damaging agent.

According to another embodiment, any combination of a tissue, cells and/or the isolated cell-derived vesicles are treated with a DNA damaging agent.

According to a specific embodiment, when eye tissue is used for isolation of cell-derived vesicles containing active wild-type p53, the eye (or part thereof) is harvested from a donor animal (e.g. animal or human) and is homogenized as to obtain cell-derived vesicles. It will be appreciated that the entire eye tissue may be used, or alternatively, a specific tissue may be selected and harvested from the eye (e.g. cornea tissue). The cell-derived vesicles are isolated by centrifugation, ultracentrifugation, filtration or ultrafiltration.

According to one embodiment, the eye cells are treated with a DNA damaging agent prior to isolation of the cell-derived vesicles. According to one embodiment, this step is performed in a tissue culture plate.

According to one embodiment, the cell-derived vesicles are first isolated and are then treated with a DNA damaging agent.

In order to improve the properties of the cell-derived vesicles against viral infection, the cell-derived vesicles may be genetically modified to further contain a peptide or polypeptide other than p53 (e.g. an immune modulator, a non-coding RNA). Such a step may be effected on a fresh batch of cell-derived vesicles or on cells from which the cell derived vesicles are obtained (e.g. on cells which were frozen and thawed).

Accordingly, the exogenous genetic material (e.g. immune modulator, non-coding RNA genetic material) can be introduced into the cell-derived vesicles by a various techniques. For example, the cell-derived vesicles may be loaded by electroporation or the use of a transfection reagent. Despite the small size of cell-derived vesicles (e.g. typically between 20-200 nm), previous publications have illustrated that it is possible to use electroporation and transfection reagent to load the cell-derived vesicles with the exogenous genetic material including DNA and RNA (see for example European Patent No. EP2419144). Typical voltages are in the range of 20 V/cm to 1000 V/cm, such as 20V/cm to 100 V/cm with capacitance typically between 25 μF and 250 μF, such as between 25 μF and 125 g. Alternatively, conventional transfection reagent can be used for transfection of cell-derived vesicles with genetic material, such as but not limited to, cationic liposomes.

Additionally or alternatively, the cell-derived vesicles (i.e. comprising a wild-type p53) may be obtained from genetically modified cells. Accordingly, the cells (i.e. from which the cell-derived vesicles are obtained) may be genetically engineered to express additional peptides, polypeptides or heterologous moieties (e.g. binding agents e.g. for specific targeting of a target cell, as discussed below).

Various methods can be used to introduce genetic material into cells such as using expression vectors. Methods of introducing expression vectors into cells are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors, such as adenovirus, lentivirus, retrovirus, Herpes simplex I virus, or adeno-associated virus (AAV). In addition, see U.S. Pat. Nos. 5,464,764 and 5,487,992 for positive-negative selection methods.

As cell-derived vesicles are derived from a variety of different cells, cells (e.g. animal or human cells, as discussed above) may be genetically engineered with an exogenous genetic material (including DNA and RNA) for expression of a polypeptide of choice (e.g. an immune activator). These cells are then cultured for an ample amount of time to produce cell-derived vesicles (e.g. for 1, 2, 3, 4, 5, 6, 12, 24, 48, 72, 96 hours, for several days e.g. 1, 2, 3, 4, 5, 6, 7, 8, 9, 10, 12, 14, 21 or 30 days, or for several weeks e.g. 1, 2, 3, 4, 5, 6, 7, 8, 10, 12 or 14 weeks) prior to harvesting of the cell-derived vesicles.

According to some embodiments of the invention, the cell-derived vesicles are targeted to a desired cell or tissue (e.g. a virally infected cell). This targeting is achieved by expressing on the surface of the cell-derived vesicles a heterologous moiety (also referred to as binding agent) which binds to a cell surface moiety expressed on the surface of the cell to be targeted. For example, the cell-derived vesicles can be targeted to particular cell types or tissues by expressing on their surface a heterologous moiety such as a protein, a peptide or a glycolipid molecule. For example, suitable peptides are those which bind to cell surface moieties such as receptors or their ligands found on the cell surface of the cell to be targeted. Examples of suitable heterologous moieties are short peptides, scFv and complete proteins, so long as the binding agent can be expressed on the surface of the cell-derived vesicle and does not interfere with expression of the wild-type p53.

According to a specific embodiment, in case of SARS-Cov-2 infection, cells (e.g. corneal cells, testicular cells, etc.) are engineered to stably express viral spike protein. Accordingly, in addition to the delivery of wild-type p53 to the target cells expressing ACE2 receptors (e.g. lung cells) vesicles expressing the spike protein also compete with SARS-CoV-2 infection by blocking the binding of the viral spike protein to ACE2 expressing cells.

As used herein, the term “viral spike protein” when relating to coronaviruses (also referred to as S protein) refers to a protein found on the viral envelope and which plays a crucial role in penetrating host cells and initiating infection. The viral spike protein typically comprises two subunits, i.e. (1) the N-terminal S1 subunit, which forms the globular head of the S protein, recognizes and binds to host cells, and (2) the C-terminal S2 region that forms the stalk of the protein and is directly embedded into the viral envelope, and is responsible for fusing the envelope of the virus with the host cell membrane.

According to one embodiment, the viral spike protein refers to a recombinant coronavirus spike protein, or a portion thereof (i.e. capable of binding a target cell).

According to some embodiments of the invention, the cell-derived vesicles are loaded with an additional therapeutic moiety such as a drug e.g. an anti-viral drug or a toxic moiety (e.g. such a small molecule).

Determination that the cell-derived vesicles comprise specific components (e.g. wild-type active p53, phosphorylated p53, or additional components e.g. immune modulators) can be carried out using any method known in the art, e.g. by Western blot, ELISA, FACS, MACS, RIA, Immunohistochemical analysis, In situ activity assay, and In vitro activity assays. Likewise, determination that the cell-derived vesicles comprise a heterologous moiety (e.g. binding agent), a cytotoxic moiety or a toxic moiety, can be carried out using any method known in the art.

According to one embodiment, once an isolated cell-derived vesicles sample has been prepared it can be stored, such as in a sample bank or freezer (e.g. at −25° C.), e.g. cryopreserved or lyophilized, and retrieved for therapeutic purposes as necessary, alternatively, the cell-derived vesicles sample can be directly used without storing the sample.

For in vivo therapy, the cell-derived vesicles comprising wild-type p53 or compositions comprising same can be administered to the subject per se or as part of a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cell-derived vesicles comprising a wild-type p53 accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include systemic, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, intratumoral or intraocular injections.

According to one embodiment, administering comprises a route selected from the group consisting of intravenous, intra-arterial, intratumoral, subcutaneous, intramuscular, transdermal and intraperitoneal.

According to a specific embodiment, the composition is for inhalation mode of administration.

According to a specific embodiment, the composition is for intranasal administration.

According to a specific embodiment, the composition is for oral administration.

According to a specific embodiment, the composition is for local injection.

According to a specific embodiment, the composition is for systemic administration.

According to a specific embodiment, the composition is for intravenous administration.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, pulmonary tissue, airway tissues, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue, eye tissue and testicular tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulary agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. cell-derived vesicles comprising wild-type p53) effective to alleviate or ameliorate symptoms of a disorder (e.g., viral infection) or prolong the survival of the subject being treated.

According to an embodiment of the present invention, an effective amount of the cell-derived vesicles comprising wild-type p53 of the some embodiments of the invention, is an amount selected to replace nonfunctional p53 in target cells (i.e. virally infected cells) by its normal, active p53 wild-type protein.

According to an embodiment of the present invention, an effective amount of the cell-derived vesicles comprising wild-type p53 of the some embodiments of the invention, is an amount selected to promote cell cycle arrest (e.g. G1 cell cycle arrest) of target cells, i.e. virally infected cells.

According to an embodiment of the present invention, an effective amount of the cell-derived vesicles comprising wild-type p53 of the some embodiments of the invention, is an amount selected to initiate or restore apoptosis (i.e. cell apoptosis) of target cells, i.e. virally infected cells.

According to an embodiment of the present invention, an effective amount of the cell-derived vesicles comprising wild-type p53 of the some embodiments of the invention, is an amount selected to initiate or restore the innate p53 anti-viral function.

Determination of a therapeutically effective amount is well within the capability of those skilled in the art, especially in light of the detailed disclosure provided herein, as discussed in detail above.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The cell-derived vesicles comprising wild-type p53 of the invention can be suitably formulated as pharmaceutical compositions which can be suitably packaged as an article of manufacture. Such an article of manufacture comprises a label for use in treating a viral infection, the packaging material packaging a pharmaceutically effective amount of the cell-derived vesicles comprising wild-type p53.

It will be appreciated that the cell-derived vesicles comprising wild-type p53 or compositions comprising same of the present invention may be administered in combination with other known treatments, including but not limited to, pro-apoptotic agents, anti-viral drugs, anti-proliferative agents and/or any other compound with the ability to reduce or abrogate the viral infection.

Exemplary pro-apoptotic agents (i.e. apoptosis inducers) which may be used in accordance with the present invention include those which affect cellular apoptosis through a variety of mechanisms, including DNA cross-linking, inhibition of anti-apoptotic proteins and activation of caspases. Exemplary pro-apoptotic agents include, but are not limited to, Actinomycin D, Apicidin, Apoptosis Activator 2, AT 101, BAM 7, Bendamustine hydrochloride, Betulinic acid, C 75, Carboplatin, CHM 1, Cisplatin, Curcumin, Cyclophosphamide, 2,3-DCPE hydrochloride, Deguelin, Doxorubicin hydrochloride, Fludarabine, Gambogic acid, Kaempferol, 2-Methoxyestradiol, Mitomycin C, Narciclasine, Oncrasin 1, Oxaliplatin, Piperlongumine, Plumbagin, Streptozocin, Temozolomide and TW 37, and combinations thereof.

Non-limiting examples of anti-viral drugs include, but are not limited to abacavir; acemannan; acyclovir; acyclovir sodium; adefovir; alovudine; alvircept sudotox; amantadine hydrochloride; amprenavir; aranotin; arildone; atevirdine mesylate; avridine; chloroquine; cidofovir; cipamfylline; cytarabine hydrochloride; delavirdine mesylate; desciclovir; didanosine; disoxaril; edoxudine; efavirenz; enviradene; envlroxlme; famciclovir; famotine hydrochloride; fiacitabine; fialuridine; fosarilate; trisodium phosphonoformate; fosfonet sodium; ganciclovir; ganciclovir sodium; hydroxychloroquine; idoxuridine; indinavir; kethoxal; lamivudine; lopinavir; lobucavir; memotine hydrochloride; methisazone; nelfinavir; nevlrapme; penciclovir; pirodavir; remdesivir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin.

According to a specific embodiment, the anti-viral drug comprises Remdesivir.

According to a specific embodiment, the cell-derived vesicles comprising wild-type p53 or compositions comprising same of some embodiments of the present invention may be administered in combination with any one or combination of Actmera (Tocilizumab), Remdesivir, Baricitinib (e.g. such as in combination with Remdesivir), Dexamethasone, Anticoagulation drugs (e.g., Clexane, Eliquis (apixaban)), Nexium (esomeprazole), Proton-pump inhibitors (PPIs), Tavanic (Levofloxacin), Acetylcysteine, Inhaled Corticosteroid (ICS), Aerovent, Solvex (Bromhexine Hydrochloride), Sopa K (Potassium gluconate), Chloroquine (e.g. Hydroxychloroquine), Antibiotic (e.g. Azenil/Azithromycin/Zitromax, Amoxicillin/Moxypen Forte, Ceftriaxone/Rocephin).

Any of the above described agents may be administered individually or in combination, together or sequentially.

The cell-derived vesicles comprising wild-type p53 or compositions comprising same of some embodiments of the present invention may be administered prior to, concomitantly with or following administration of the latter.

According to one embodiment, there is provided a method of inducing cell cycle arrest and/or apoptosis of a virally infected cell, the method comprising contacting the cell with an effective amount of cell-derived vesicles comprising wild-type p53.

The term “apoptosis” as used herein refers to the cell process of programmed cell death. Apoptosis characterized by distinct morphologic alterations in the cytoplasm and nucleus, chromatin cleavage at regularly spaced sites, and endonucleolytic cleavage of genomic DNA at internucleosomal sites. These changes include blebbing, cell shrinkage, nuclear fragmentation, chromatin condensation, and chromosomal DNA fragmentation. Furthermore, apoptosis produces cell fragments called apoptotic bodies that phagocytic cells are able to engulf and quickly remove before the contents of the cell can spill out onto surrounding cells and cause damage. Furthermore, phagocytic cells are capable of presenting viral particles to other immune cells in order to activate the immune system against the virus.

The term “cell cycle arrest” refers to the cell's state when it is prevented from progressing through the next phase of the cell cycle, e.g. from the G1 stage to S stage, from S stage to G2 stage, or from G2 stage to M stage.

According to one embodiment, the cell cycle arrest is a G1 cell cycle arrest.

The term “G1 cell cycle arrest” refers to the cell's state when it is prevented from progressing from the G1 stage to the S stage. Typically G1 cell cycle arrest occurs in response to diverse governing conditions (DNA damage, contact inhibition, growth factors, viral infection, etc.) that control cellular progress through the G1 phase of the cell cycle. G1 progress is controlled by the phosphorylation state of cyclin/CDK complexes.

According to one embodiment, induction of G1 arrest prevents transcription of viral proteins.

According to one embodiment, the method of contacting the cell-derived vesicles comprising wild-type p53 of the present invention with the target cells, i.e. virally infected cells, is effected in-vivo.

According to one embodiment, the method of contacting the cell-derived vesicles comprising wild-type p53 of the present invention with the target cells, i.e. virally infected cells, is effected ex-vivo. Ex vivo treatments are well known in the art and include, without being limited to, apheresis and leukapheresis.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Cells

Vero E6 cells (Monkey kidney epithelial cells) were maintained in Dulbecco's modified Eagle's medium (DMEM; Lonza), supplemented with 8% fetal calf serum (FCS; Bodinco), 2 mM L-glutamine, 1% Penicillin/Streptomycin (Sigma-Aldrich).

GBM LN-18 cells were maintained in Dulbecco's modified Eagle's medium (DMEM; Lonza), supplemented with 5% fetal bovine serum (FBS; Bodinco), 2 mM L-glutamine, 1 mM sodium pyruvatel % Penicillin/Streptomycin (Sigma-Aldrich).

Viruses

The clinical isolate SARS-CoV-2/Leiden-0002 was isolated from a nasopharyngeal sample. Additionally, Alpha-coronavirus (CoV 229E) and Beta-coronavirus (SARS-Cov2, SARS-Cov2 and CoV OC43) are used for testing efficiency of treatment.

Active Agents

Eyes of male Sprague Dawley (SD) rats were harvested from already sacrificed animals. Cornea was dissected from eye tissue, incubated in culture medium and UV irradiated. Corneal epithelium was dissected from cornea and homogenated (as described below). Alternatively, cell-derived vesicles were first harvested from corneal homogenates and were then subjected to UV irradiation inducing p53 phosphorylation (in the cell-derived vesicles).

Chicken eyes were obtained from sacrificed animals. Eyes were kept in ice until use. Chicken eye tissue was obtained and corneal epithelia were used as a source for corneal homogenates to obtain native cell-derived vesicles containing p53 (as described below). Chicken cornea was induced by UV irradiation, homogenate and cell-derived vesicles were harvested (as described below). Alternatively, cell-derived vesicles were first harvested from corneal homogenates and were then subjected to UV irradiation inducing p53 phosphorylation in the cell-derived vesicles.

Similarly, swine eyes are obtained from sacrificed animals. Eyes are kept in ice until use. Swine eye tissue is obtained and corneal epithelia is used as a source for corneal homogenates to obtain native cell-derived vesicles containing p53 (as described below). Swine cornea is induced by UV irradiation, homogenate and cell-derived vesicles are harvested (as described below). Alternatively, cell-derived vesicles are first harvested from corneal homogenates and are then subjected to UV irradiation inducing p53 phosphorylation in the cell-derived vesicles.

Alternatively, culture medium and corneal homogenate is obtained from available human corneal epithelial cell lines (e.g. HCE from Episkin). P53 phosphorylation is induced by UV irradiation of cell lines. Following irradiation, cell-derived vesicles are harvested.

Furthermore, other tissues including, skin (epidermis), testis (gonads), brain structures, and the epithelium of the small intestine are used as a source for native cell-derived vesicles containing p53. Cell-derived vesicles are obtained from these tissues in the same manner as for eye tissue.

For example, UV irradiation is carried out by irradiation with a UV lamp (312 nm) at 150 mJ/cm2. The tissue or cells (e.g. in a petri dish) is placed 15-30 cm above a UV light source (e.g. 4×6 W, 312 nm tube, power 50 W, TFP-10M, Vilber Lourmant, Torcy, France) for 5-15 minutes. The UV dosimetry is performed using a UV light meter (YK-34UV; Lutron Electronic, Taiwan).

p53-comprising cell-derived vesicles (also referred to herein as EXO or EXO_002), were obtained from rat and chicken cornea, respectively, as follows:

Isolation of Cell-Derived Vesicles

Isolation of cell-derived vesicles was performed from both tissue/cell homogenate and from culture medium after cell cultivation.

Homogenate Preparation and Isolation of Cell-Derived Vesicles from Tissues/Cells

Ultracentrifugation Method

Tissues \cells were added to a Teflon grinder and homogenized in minimal needed volume of culture medium. Initial centrifugation (e.g., 10,000×g for 10 min) was used which separates cells and cell detritus from supernatants. After centrifugation, the pellets were discarded and the supernatants (optional) were passed through a filter 0.2 μm. The supernatants were collected and loaded on top of a 40% sucrose solution and second centrifugation was carried out (e.g., at 100,000×g for 1 hour). Due to their density, cell-derived vesicles (e.g. exosomes) enter the sucrose solution. The sucrose solution was harvested, diluted with PBS or culture medium and centrifuged again (e.g., at 100,000×g for 1 hour) to pellet the cell-derived vesicles (e.g. exosomes). The resultant exosomal pellets were re-suspended in McCoy 5A culture medium.

Precipitation Method—ExoQuick™

This method is carried out according to the manufacturer's instructions (System Biosciences). Briefly, culture medium of corneal epithelium cells lines or corneal epithelium cell homogenate was diluted in PBS and mixed with of ExoQuick-TC™ solution by inverting the tube several times. The sample was incubated at 4° C. then centrifuged twice (e.g., at 1,500×g for 30 and 5 minutes, respectively), in order to remove the supernatant. The supernatant was discarded, and the pellet was re-suspended in PBS.

Freezing Procedure of Cell-Derived Vesicles

Cell-derived vesicles obtained from chicken or rat cornea, as described above, were stored frozen for about one year at −25° C. Prior to use, the cell-derived vesicles were thawed in 1.5 ml eppendorf tubes for about 1 hour at 4° C.

Culture of p53-Comprising Cell-Derived Vesicles and Virally Infected Cells

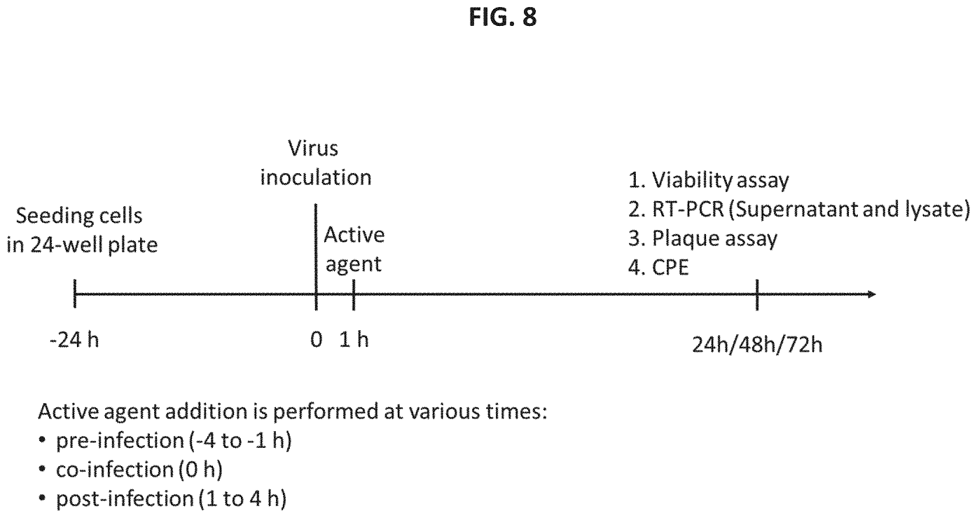

Vero E6 cells were plated at required density (for example at a density of 4×10⁴/well) in 24-well plates with appropriate controls as presented in Table 2, below. 24 hours later, the cells in some wells (as illustrated in FIG. 8) are infected in the biosafety level 3 laboratory with SARS-CoV2 at an MOI of 0.01. The inoculum is removed after 1 hour (post-infection option) and replaced by fresh medium complemented with different concentrations of the p53-comprising cell-derived vesicles obtained from corneal cells (at compound concentrations between 0.1% and 50% of total medium volume, wherein the particle concentration is typically between 2.48×10¹² and 1.40×10¹⁰ particles/ml). XTT assay was performed in parallel (as discussed below), to measure virus cytopathic effect under particular culture conditions.

TABLE 2
experimental conditions for in vitro experiment

| virus infection | Exosome treatment |
|---|---|
| − | − |
| + | − |
| + | + |
| − | + |

Assessment of Viral Load by RT-PCR

Virus RNA concentration in supernatant and cell lysate are measured by real-time PCR (RT-PCT) during the exponential growth phase of the virus (24 hrs, 48 hrs, and 72 hrs). During this time, the viruses typically exhibit growth by several orders of magnitudes if no inhibitor was added. For RNA preparation, the method previously described in Bloom et al. is utilized [Bloom et al., J Clin Microbiol (1990) 28(3):495-503]. Quantitative real-time PCR assays are performed with the purified RNA based on previously published protocols [Gibb et al., Mol Cell Probes (2001) 15(5):259-66; Drosten et al., N Engl J Med (2003) 348:1967-1976; Asper et al., Journal of Virology (2004) 78(6):3162-3169; Gunter et al., Antiviral Res. (2004) 63(3):209-15]. In vitro transcripts of the PCR target regions are amplified using PCR to generate standard curves for quantification of the virus RNA in supernatant and lysate. Concentrations of p53-comprising cell-derived vesicles required to inhibit virus replication by 50% (IC50) or 90% (IC90) are calculated.

XTT Assay (2,3-bis-(2-methoxy-4-nitro-5-sulfophenyl)-2H-tetrazolium-5-carboxanilide)

Cell viability (due to growth inhibition or cytotoxicity) was evaluated by the enzymatic XTT assay analysis (kit was obtained from Sigma, Israel).

XTT was used to assess cell viability as a function of redox potential. This assay produces a water-soluble orange-colored formazan product which dissolves directly into the culture medium. Its concentration determined by optical density.

Cell viability was assessed 24, 48 and 72 hours following treatment with p53-comprising cell-derived vesicles obtained from obtained from corneal cells. Specifically, cell growth medium was replaced by fresh media (100 μl per well) containing 1 mg/ml XTT and incubated for 2 hours or 4 hours. Absorbance at 490 nm and reference wavelength of 690 nm were recorded on an automated microplate reader.

Cytopathic Effect (CPE) Reduction Assay

Cells (e.g. Vero E6 cells) were seeded in 96-well cell culture plates at a density of 10⁴ cells per well. Twenty-four hours later cells were either mock-infected (analysis of cytotoxicity of the compound) or were infected with 300 PFU of SARS-CoV-2 virus per well (MOI of 0.015) in a total volume of 150 μl of medium with compound. Subsequently, 1 hr later, different dilutions of the p53-comprising cell-derived vesicles obtained from corneal cells were added (in triplicates), as follows: 1:4, 1:8, 1:16, 1:32, 1:64, 1:128, 1:256, 1:512. The highest dose tested was a 1:4 dilution (i.e. 25% of medium volume was p53-comprising cell-derived vesicles). Cell viability was assessed three days post-infection by XTT assay (Sigma, Israel) and absorption was measured at 495 nm with an EnVision Multilabel Plate Reader (PerkinElmer).

Plaque Assay

All samples are serially diluted in sterile PBS. Plaque assays are performed using Vero E6 cells at confluency in 6-well cell culture plates. Briefly, plates are washed with sterile PBS. All samples are then plated in duplicates at 100 μL per well. Plates are incubated at 37° C. for 45 minutes with occasional rocking. Then 2 mL of 0.5% agarose in minimal essential media (MEM) containing 2% FBS and antibiotics is added per well. Plates are incubated at 37° C. for 24/48/72 hours. The cells are fixed with 10% buffered formalin, followed by the removal of the overlay, and then stained with 0.2% crystal violet to visualize plaque forming units (PFU). All assays are performed in BSL-3 laboratory setting

Image Immunofluorescence

Infected Vero E6 cells are fixed at the indicated time post-infection with 5% paraformaldehyde for 4 hours and permeabilized with 0.5% Triton X-100 for 5 min. After blocking with 3% bovine serum albumin (BSA) for 30 min, the cells are incubated for 1 hour at room temperature with rabbit anti-SARS-CoV nucleoprotein (NP) serum, which exhibits strong cross-reactivity with SARS-CoV-2 NP. After two washes with PBS the cells are incubated with Alexa Fluor 488-conjugated goat-anti-rabbit IgG (Thermo Fisher Scientific) for 1 hour at room temperature. After two additional washes, PBS supplemented with 0.1m ml⁻¹ DAPI (BioLegend) is added to the cells for at least 30 min before imaging. Images are acquired using the Celigo Image Cytometer (Nexcelom). The assay results and data analysis enable to determine infectivity and viability or cytotoxicity. On the basis of all infectivity and cytotoxicity values, a four-parameter logistic nonlinear regression model is used to calculate EC50 and 50% cytotoxic concentration (CC50) values whenever required.

AnnexinV/PI Staining

LN-18 cells were harvested and counted manually. Cells were seeded in a 24-well plate at 30000 cells per well and allowed to adhere overnight.

The following day, p53-comprising cell-derived vesicles obtained from corneal cells were added.

Cell count and apoptosis assay were performed 24 hours after incubation with Exo using Annexin-PI staining detected by FACS according to the manufacturer instructions

In Vivo Toxicity Assay

ICR mice are injected with the cell-derived vesicles (e.g. obtained from corneal cells) either in a therapeutic concentration or in an escalated dose concentration, 250 μl and 500 μl, respectively. Each mouse receives treatment daily by intraperitoneal injection as follows:

Group 1: 4 mice—250 μl

Group 2: 4 mice—500 μl

Group 3: 3 mice, naïve, no treatment (Control group)

Animals are inspected for reaction to treatment 1, 4, 8, and 24 hours after each injection. No abnormal signs in animal appearance, behavior, food consumption, stool, or irritation at the injection site are expected. Body weight is measured daily during the study. Body weight range is expected to remain normal within the treatment groups (with no significant gain or loss of body weight). 24 hours after the last injection (study day 8), blood is collected for count and chemistry, and mice are sacrificed with CO₂. Main organs: heart, lungs, liver, spleen, kidneys, pancreas and brain are isolated for macroscopic examination and weighing. No macroscopic lesions or abnormalities are expected to be detected. All results are expected to be within normal range.

Transgenic Mice Study

K18-ACE2 transgenic mice (Jackson labs) are challenged i.n. (intra nasally) with 1×10⁵ PFU of SARS-CoV-2 virus on day 0. The mice are also treated with p53-comprising cell-derived vesicles (e.g. obtained from corneal cells) by i.n. administration (of about 2.48×10¹² particles/ml in 1 μl) or by intraperitoneal injection (i.p.) (of about 2.48×10¹² particles/ml in 200 μl). One group of mice is treated by p53-comprising cell-derived vesicles i.p. once (or twice) daily on days −1, 0, 1, 2, 3. Another group is treated by i.n. treatment of p53-comprising cell-derived vesicles once (or twice) daily on days −1, 0, 1, 2, 3.

Lungs of selected mice from each group are harvested on day 3 and viral titers are determined by serial dilutions onto 96-well MDCK plates. Mice survival rate is monitored until day 12 after infection.

Macaque Studies

Three groups of cynomolgus macaques are infected intratracheally with 1×10⁶ TCID50 SCV2 suspended in 5 ml of PBS. The control group (n=4) receives wet inhalation (W.I.) with PBS, while the prophylactic (n=6) and postexposure (n=4) groups are treated with p53-comprising cell-derived vesicles (e.g. obtained from corneal cells) W.I. The prophylactic group is treated on days −1, 0, 1, 2, and 3 after SCV2 infection; the postexposure group is treated on days 0, 1, 2, and 3 after SCV infection.

At days −1, 0, 1, 2, 3, and 4, the macaques are anesthetized with ketamine, 10 ml of blood is collected from inguinal veins and pharyngeal swabs are taken, which are placed in 1 ml transport medium. Pharyngeal swabs are frozen at −70° C. until RT-PCR analysis (as discussed above). One lung of each macaque is inflated with 10% neutral-buffered formalin by intrabronchial intubation and suspended in 10% neutral-buffered formalin overnight. Samples are collected in a standard manner (one from the cranial part of the lung, one from the medial part and two from the caudal part), embedded in paraffin, cut at 5 μm and used for immunohistochemistry (as discussed below) or stained with H&E (as discussed below).

For semiquantitative assessment of SCV2 infection—associated inflammation in the lung, each H&E-stained section is examined for inflammatory foci by light microscopy using a 10× objective. Each focus is scored for size (1, smaller than or equal to area of 10× objective; 2, larger than area of 10× objective and smaller than or equal to area of 2.5× objective; 3, larger than area of 2.5× objective) and severity of inflammation (1, mild; 2, moderate; 3, marked). The cumulative scores for the inflammatory foci provide the total score per animal. Sections are examined without knowledge of the identity of the macaques.

Three lung tissue samples taken from the other lung (one from the cranial part of the lung, one from the medial part and one from the caudal part) are homogenized in 2 ml transport medium using Polytron PT2100 tissue grinders (Kinematica). After low-speed centrifugation, the homogenates are frozen at −70° C. until inoculation on Vero 118 cell cultures in tenfold serial dilutions. The identity of the isolated virus are confirmed as SCV2 by RT-PCR of the supernatant.

Immunohistochemistry

The same formalin-fixed, paraffin-embedded lung samples that are used for histology—one from the cranial part of the lung, one from the medial part and two from the caudal part—are cut at 5 μm and stained for SCV2 antigen using either biotinylated purified human IgG from a convalescent SARS-Cov-2 patient, negative-control biotinylated purified human IgG, or the dilution buffer, as described previously (Kuiken et al., Lancet (2003) 362(9380):263-70). Twenty-five arbitrarily chosen 20× objective fields of lung parenchyma in each lung section are examined by light microscopy for the presence of SCV2 antigen expression, without knowledge of the identity of the macaques. The cumulative scores for each animal are expressed as number of positive fields per 100 fields (%). Selected lung sections from macaques in the control group are stained with monoclonal antibody AE1/AE3 to cytokeratin (Neomarkers) for identification of epithelial cells, according to standard immunohistochemical procedures.

Hematoxylin and Eosin (H&E) Staining

H&E stained tissue sections are used for anatomical pathology diagnosis. The H&E procedure stains the nucleus and cytoplasm contrasting colors to readily differentiate cellular components.

### Example 1

### Efficacy and Specificity of p53 Comprising Cell-Derived Vesicles on Cancer Cells

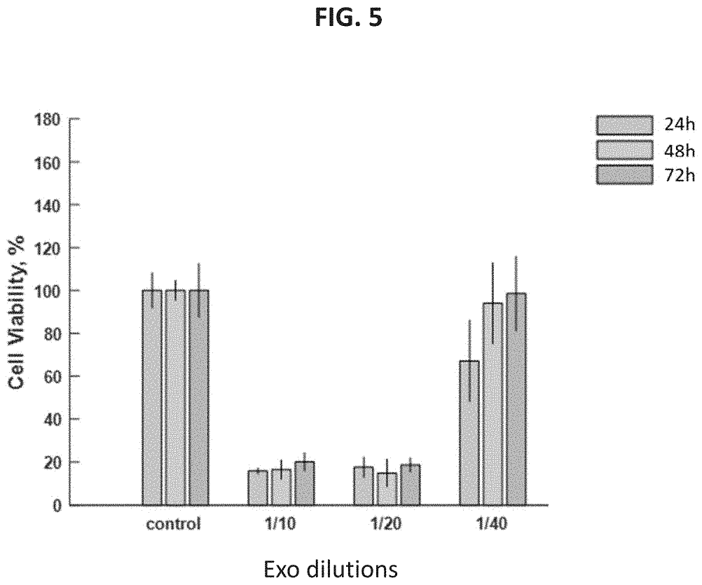

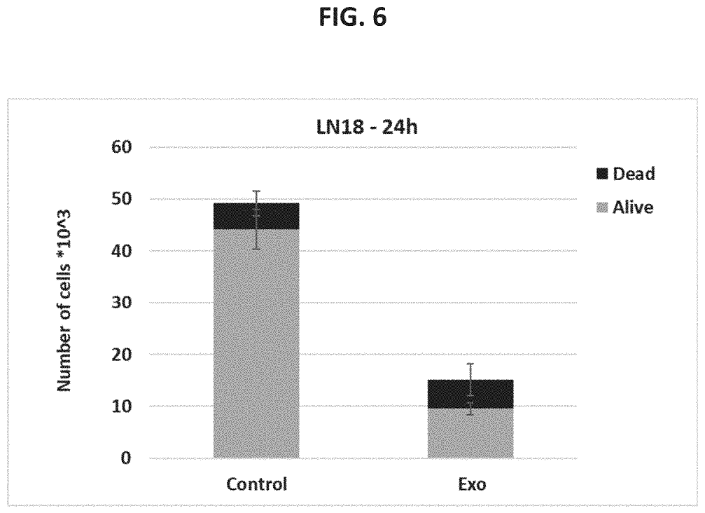

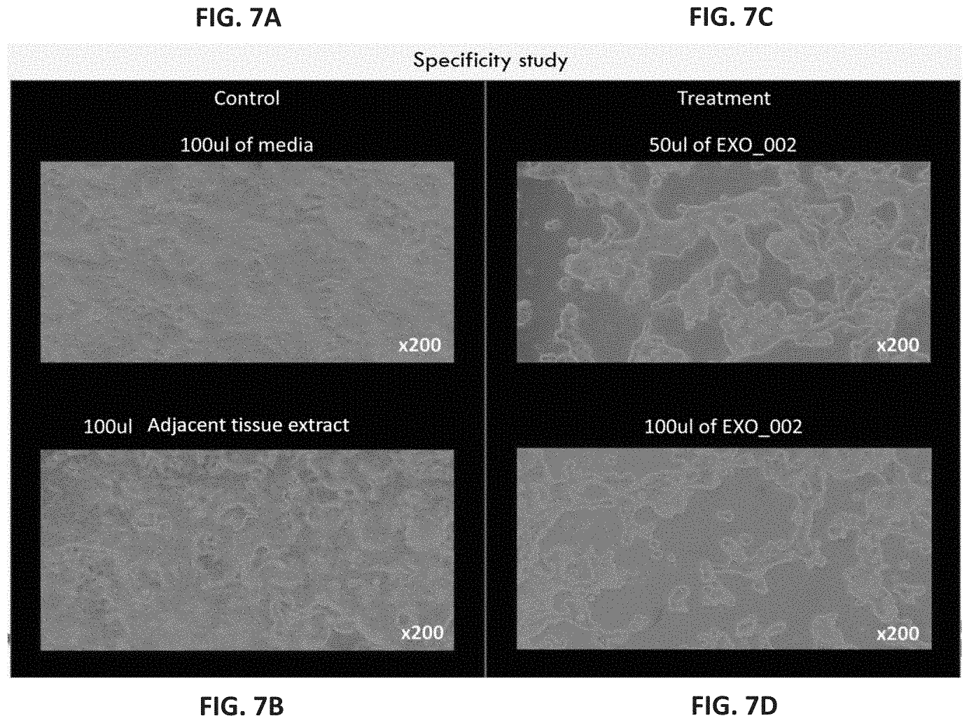

Preliminary studies illustrated the anti-cancer effect of wild-type p53 comprising cell-derived vesicles on malignant cells. Specifically, FIGS. 5-6 illustrate the activity and dose effect of p53-comprising cell-derived vesicles on p53 mutated human glioblastoma cell line LN-18. These results support the theory that p53-comprising cell-derived vesicles are capable of effectively penetrating cells and have a therapeutic effect (FIGS. 5-6). FIGS. 7A-D illustrate comparative effect of p53-comprising cell-derived vesicles obtained from corneal cells as opposed to vesicles harvested from different tissues (adjacent to cornea). While administration of p53-comprising cell-derived vesicles obtained from corneal cells leads to substantial cell death (FIGS. 7C-D), administration of vesicles derived using the same protocol from a proximal tissues did not suppress cell growth and lead to results identical to control (FIGS. 7A-B).

### Example 2

### In-Vitro Proof of Concept of the Antiviral Efficacy of p53-Comprising Cell-Derived Vesicles

The antiviral effect of wild-type p53-comprising cell-derived vesicles obtained from corneal cells is evaluated using Vero E6 cells infected by SARS Cov2 virus. Supernatant and lysate RT-PCR, Plaque assay and/or Image Immunofluorescence based antiviral testing are performed 24/48/72 hours after virus inoculation. Cytopathic effects (CPE) are examined with an optical microscope. These assays are performed to illustrate that the viral load in p53-comprising cell-derived vesicles-treated cultures is significantly lower than untreated control cultures in a dose dependent manner.

In parallel cell viability assay is performed to show absence of p53-comprising cell-derived vesicles cytotoxicity and base viral cytotoxicity is evaluated for further comparison. The testing paradigm is presented in FIG. 8.

### Example 3

### Antiviral Efficacy of p53-Comprising Cell-Derived Vesicles on SARS-CoV-2

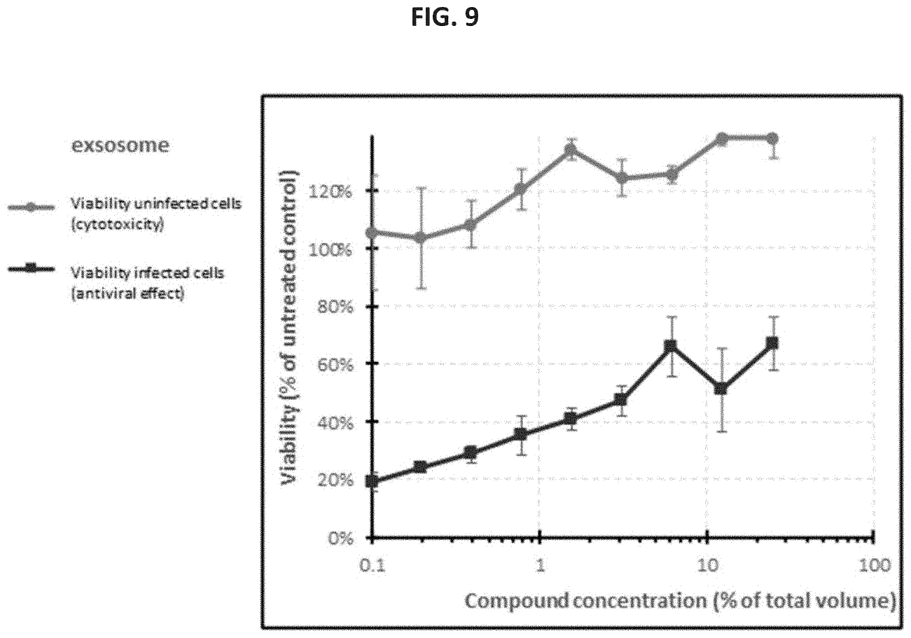

As illustrated in FIG. 9, p53-comprising cell-derived vesicles obtained from corneal cells showed a positive effect on the survival of SARS-CoV-2 infected Vero E6 cells. Virally infected cells treated with the highest dose of p53-comprising cell-derived vesicles showed significantly higher viability (70%) as compared to untreated virally infected cells (20%) (FIG. 9).

These initial results demonstrated an anti-viral activity of p53-comprising cell-derived vesicles obtained from corneal cells in combination with complete lack of toxicity even in the highest doses tested (FIG. 9).

Reduction of viral load (by RT-PCR of cell culture supernatants and cell lysates) and of viral titration are expected. Furthermore, higher concentrations of the active compound is expected to show a further improvement in viability of the virally infected cells.

### Example 4

### In-Vivo Proof of Concept of the Antiviral Efficacy of p53-Comprising Cell-Derived Vesicles

The antiviral efficacy of wild-type p53-comprising cell-derived vesicles (e.g. obtained from corneal cells) is assessed in vivo in K18-ACE2 transgenic mice (Jackson labs) as well as in the non-human primate (NHP) model of Macaques—SARS-CoV and SARS-CoV-2.

As discussed above (see ‘General Materials and Experimental Procedures’ section), transgenic mice (Jackson labs) are challenged i.n. (intra nasally) with SARS-Cov-2 virus (LD50 influenza H1N1 A/PR/8/34) on day 0. One group of mice is treated by p53-comprising cell-derived vesicles via intraperitoneal injection (i.p.) once (or twice) daily on days −1, 0, 1, 2, 3. Another group is treated by i.n. (intra nasal) treatment of p53-comprising cell-derived vesicles once (or twice) daily on days −1, 0, 1, 2, 3.

Lungs of selected mice from each group are harvested on day 3 and viral titers are determined. Mice survival rate is monitored until day 12 after infection. Treated mice are expected to have significantly reduced morbidity as compared to untreated control mice.

Similarly, Macaques are infected intratracheally with tissue-culture infectious doses (TCID50) of SARS-CoV-2 (as previously taught by Shan et al., Cell Research (2020) 30: 670-677). The control group (n=4) receives wet inhalation (W.I.) with PBS, while the prophylactic (n=6) and post-exposure (n=4) groups are treated with p53-comprising cell-derived vesicles W.I. The prophylactic group is treated on days −1, 0, 1, 2, and 3; the post-exposure group is treated on days 0, 1, 2, and 3.

At days −1, 0, 1, 2, 3, and 4, blood is collected from inguinal veins and pharyngeal swabs are taken. Lungs from each macaque is collected for further analysis of inflammation and viral titer. Macaque survival rate is monitored until day 12 after infection. Treated Macaques are expected to have significantly reduced morbidity as compared to untreated control Macaques.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of treating a viral infection in a subject in need thereof, the method comprising administering to the subject a therapeutically effective amount of cell-derived vesicles comprising wild-type p53, thereby treating the viral infection in the subject.

2. (canceled)

3. A method of inducing cell cycle arrest and/or apoptosis of a virally infected cell, the method comprising contacting said cell with an effective amount of cell-derived vesicles comprising wild-type p53.

4. (canceled)

5. The method according to claim 1, wherein said viral infection is caused by a RNA virus.

6. The method according to claim 1, wherein said viral infection is caused by a DNA virus.

7. (canceled)

8. The method according to claim 1, wherein said viral infection is caused by a Coronavirus.

9-11. (canceled)

12. The method according to claim 1, wherein said cell-derived vesicles comprise exosomes.

13. The method according to claim 1, wherein said cell-derived vesicles are essentially devoid of intact cells.

14-15. (canceled)

16. The method according to claim 1, wherein said cell is a cell of an animal or a human eye tissue.

17-19. (canceled)

20. The method according to claim 16, wherein said eye tissue comprises a corneal epithelium tissue.

21. The method according to claim 20, wherein said corneal epithelium tissue comprises corneal epithelial cells.

22-23. (canceled)

24. The method according to claim 1, wherein said cell is a healthy cell.

25. The method according to claim 1, wherein said cell is a genetically non-modified cell.

26-27. (canceled)

28. The method according to claim 1, wherein said cell has been treated with a DNA damaging agent to activate said wild-type p53 protein.

29. (canceled)

30. The method according to claim 1, wherein said wild-type p53 comprises phosphorylated wild-type p53.

31. The method according to claim 1, wherein an outer surface of said cell-derived vesicles comprise a heterologous moiety for targeted delivery of said cell-derived vesicles to a target cell.

32-37. (canceled)

38. The method according to claim 1, wherein the subject is a human subject.
